(19) United States
(12) Patent Application Publication
(10) Pub. No.: US 2023/0056311 A1
(43) Pub. Date: Feb. 23, 2023

(54) METHOD FOR IMPROVING PRODUCTION OF STREPTOMYCES POLYKETIDE COMPOUNDS

(71) Applicants: EAST CHINA UNIVERSITY OF SCIENCE AND TECHNOLOGY, Shanghai (CN); INSTITUTE OF MICROBIOLOGY, CHINESE ACADEMY OF SCIENCES, Beijing (CN)

(72) Inventors: Lixin ZHANG, Shanghai (CN); Weishan WANG, Beijing (CN); Shanshan LI, Beijing (CN); Zilong LI, Beijing (CN); Gaoyi TAN, Shanghai (CN); Jingyu ZHANG, Shanghai (CN)

(21) Appl. No.: 17/611,992

(22) PCT Filed: May 15, 2020
(86) PCT No.: PCT/CN2020/090452

§ 371 Date: Nov. 17, 2021

(30) Foreign Application Priority Data
May 17, 2019 (CN) 201910411123.7

(51) Int. Cl. C12P 13/00; C12N 15/01; C12N 1/20; C12N 9/02; C12N 9/10; C12N 9/16; C12P 17/16; C12P 17/18

(52) U.S. Cl. CPC C12P 13/001; C12N 15/01; C12N 1/205; C12N 9/001; C12N 9/1029; C12N 9/16; C12P 17/162; C12P 17/18; C12Y 103/01008; C12Y 203/01086; C12Y 301/02001; C12R 2001/465

(57) ABSTRACT

A method for improving the production of Streptomyces polyketide compounds is provided. The method greatly improves the capability of the Streptomyces polyketide compounds by strengthening a triacylglycerol decomposition pathway in Streptomyces during the stationary phase. A method for switching the primary metabolism of Streptomyces to the secondary metabolism, Streptomyces producing polyketide compounds, and use thereof are also provided.

### CROSS REFERENCES TO RELATED APPLICATIONS

This application claims priority benefits to Chinese Patent Application No. 201910411123.7 filed with the National Intellectual Property Administration of the People's Republic of China on May 17, 2019. The contents of all of the aforementioned application are all incorporated herein by reference.

Sequence Listing Statement

The ASCII file, entitled SHP211121US_Sequence Listing.txt, created on Sep. 13, 2022, comprising 5,362 bytes, submitted concurrently with the filing of this application is incorporated herein by reference.

### TECHNICAL FIELD

The present disclosure relates to the field of microbial engineering. More specifically, the present disclosure improves the production of polyketide compounds from engineered bacteria by improving the decomposition of triacylglycerol (TAG) in engineered Streptomyces during a stationary phase of fermentation.

### BACKGROUND

Polyketide compounds are a class of secondary metabolites synthesized by organisms, and they are usually organic substances with high biological activity. Many drugs in clinic use, such as antibiotics, immunosuppressants, antiparasitics, and antineoplastic agents, are naturally synthesized or derived polyketide compounds.

Industrial Streptomyces is the most important engineered bacteria for producing polyketide compounds by fermentation. In the initial stage of fermentation, bacteria consume external nutrients and grow rapidly. When essential nutrients become limited, Streptomyces undergoes a metabolic switching from primary metabolism to secondary metabolism (Nieselt K. et al., BMC Genomics 11, 10, 2010), and begins to synthesize secondary metabolites (such as polyketide compounds) (Bibb M. J et al., Curr Opin Microbiol 8,208-215, 2005; Alam M et al., BMC Genomics 11, 202, 2010). Carbon sources in the culture medium are almost depleted at this time. Although substantial progress has been made in the research on polyketone biosynthetic pathway and its regulation pathway in the art, carbon sources used in polyketone synthesis pathway, that is, sources of intracellular direct precursors, still remain unknown after the exogenous carbon sources are consumed.

### SUMMARY OF THE INVENTION

According to the present disclosure, metabolic flux in the biosynthesis process of polyketide compounds is dynamically analyzed for the first time, and it is proposed that the production of polyketide compounds can be improved by strengthening a triacylglycerol (TAG) decomposition pathway in Streptomyces during a stationary phase.

In a first aspect, the present disclosure provides a method for improving the production of a polyketide compound in a Streptomyces, comprising a step of strengthening a triacylglycerol decomposition pathway in a Streptomyces, preferably the Streptomyces during a stationary phase.

In a second aspect, the present disclosure provides a method for switching a primary metabolism to a secondary metabolism in a Streptomyces, comprising strengthening a triacylglycerol decomposition pathway in the Streptomyces.

In a third aspect, the present disclosure provides a Streptomyces for producing a polyketide compound by fermentation, wherein an expression level and/or activity of at least one enzyme in the Streptomyces that catalyzes an irreversible reaction of a β-oxidation pathway is enhanced compared with an original strain.

In a fourth aspect, the present disclosure provides a use of the Streptomyces of the third aspect in the production of a polyketide compound by fermentation.

### Technical Effects

Most microorganisms start secondary metabolism when nutrients are depleted. Therefore, a better understanding of the turning-on and switching pathways of secondary metabolism (for example, switching from an extracellular carbon source to an intracellular carbon source) is of great significance for fermentation engineering based on secondary metabolism. Direct carbon sources for biosynthesis of polyketide compounds after depletion of external nutrients remain unknown. The experimental results of this study demonstrate that an intracellular TAG pool provides precursors for polyketide biosynthesis during a stationary phase (i.e., provides a carbon source) and regulates direction of metabolic flux. Specifically, since a large amount of NADH with reducing power is generated via a fatty acid β-oxidation pathway during the decomposition of TAG, and the activities of citrate synthase, isocitrate dehydrogenase and α-ketoglutarate dehydrogenation in the TCA cycle are repressed by high level of reducing power, the metabolic flow at the node of acetyl-coenzyme A to TCA is thus reduced, thereby enhancing the flow to polyketide biosynthesis. This is completely different from previous studies in which TAG pathway is only a competitive pathway for polyketide biosynthesis (Craney, A. et al., Chem Bio119, 1020-1027 (2012); Zabala, D. et al., Metab Eng 20, 187-197 (2013)).

Although changes in the transcription profile during the switching from primary metabolism to secondary metabolism have been revealed in the field (Nieselt, K., et al., BMC Genomics 11, 10 (2010); Liu, G, et al., Microbiol Mol Biol Rev 77, 112-143 (2013); Bibb, M J, et al., Curr Opin Microbiol 8, 208-215 (2005)), it is not yet possible to account for a decrease in the activity (post-translational level) of primary metabolic enzymes during the stationary phase. Our research found that a high NADH/NAD⁺ ratio during the stationary phase can inhibit citrate synthase, isocitrate dehydrogenase and α-ketoglutarate dehydrogenase, which in turn leads to a decrease in carbon flux to TCA cycle. In particular, the inventors found that this high NADH/NAD⁺ ratio is mainly caused by TAG decomposition in view of the fact that β-oxidation provides a large amount of reducing equivalents (NADH and FADH). Therefore, the decomposition of TAG during the stationary phase has a dual effect: it not only provides precursors (carbon sources) and energy (reducing power) for the synthesis of polyketide compounds, but also regulates the redistribution of carbon flux. The result of the present disclosure provides a basic mechanism for dynamic change of carbon metabolic flux during the whole fermentation process, and proposes a new mechanism for switching from primary metabolism to secondary metabolism in Streptomyces.

Based on this mechanism, the present disclosure proposes to increase the production of polyketide compounds from engineered Streptomyces through temporal regulation of TAG decomposition. As proved by the embodiments, the regulation method in the present disclosure has wide applicability in Streptomyces: on a laboratory scale, amount of actinorhodin produced by Streptomyces coelicolor is increased by 190%, amount of jadomycin B produced by Streptomyces venezuelae is increased by 170%, and amount of oxytetracycline produced by Streptomyces rimosus is increased by 47%, which reaches 9.17 g/L; and on an industrial fermentation scale, amount of abamectin B1a is increased by 50%, which reaches 9.31 g/L. It is the highest production currently reported available on an industrial scale. The strategy of enhancing TAG decomposition during the stationary phase proposed by the present disclosure provides a new solution for the fermentation improvement of polyketide compounds.

### BRIEF DESCRIPTION OF THE DRAWINGS

FIG. 1 shows the results of analysis of metabolite pools using time-course comparative metabolome. (a) Schematic of the switching from primary metabolism to secondary metabolism showing the trend of polyketide production, bacterial biomass and carbon sources in liquid medium over time. (b) Changes in glucose consumption, bacterial biomass, and actinomycin (Act) production during the fermentation process of Streptomyces coelicolor M145 and HY01 over time. (c) Identification and classification of the metabolites of M145 by GC-MS analysis. TCA: tricarboxylic acid cycle; NUM: nucleotide metabolism; FMM: fructose and mannose metabolism; EMP: glycolysis; AAM amino acid metabolism; GM: galactose metabolism; GDM: glyoxylic acid and dicarboxylic acid metabolism; PPP: pentose phosphate pathway; LPM: lipid metabolism; PGI: mutual conversion of pentose and glucuronic acid. (d) Changes of metabolites levels in different metabolic pathways in M145 over time. The amounts of metabolites at 20 h are normalized as one. Significance is analyzed by t-test (p<0.05 is considered statistically significant, and the significance levels are ***p<0.001, **p<0.01 and *p<0.05, ns means it is not significant). (e) Changes in phospholipid (PL) and TAG levels in M145 over time. The amounts of metabolites at 20 h are normalized as one. (f) Proportion of different fatty acid moieties of TAGs in M145 cultured at 48 h. TAG-iC13:0: isotridecanoate; TAG-aiC13:0: anteisotridecanoate; TAG-iC14:0: isomyristate; TAG-C14:0: myristate; TAG-iC15:0: isopentadecanoate; TAG-aiC15:0: anteisopentadecanoate; TAG-C15:0: pentadecanoate; TAG-iC16:0: isopalmitate; TAG-C16:0: palmitate; TAG-iC17:0: isostachidecanoate; TAG-aiC17:0: anteisomargarate; TAG-C17:0: heptadecanoate; TAG-C18: stearate; TAG-C16:1-Δ9: 9-hexadecenoic acid; TAG-C18:1-Δ9: 9-octadecenoic acid. Data shown in Fig. (b) are the average and standard deviation (s.d.) of three independent experiments. Data shown in Figs. (d) and (e) are the average and s.d. of five independent experiments.

FIG. 2 demonstrates that intracellular TAG pool contributes to polyketide biosynthesis during the stationary phase. (a) Changes in concentrations of different fatty acid moieties from intracellular TAG pools between 48-96 h strains M145 and HY01. (b) Top 20 metabolites with significant change in intracellular concentration between M145 and HY01 during the stationary phase. AcCoA: acetyl-coenzyme A; MalCoA: malonyl-coenzyme A; DHAP: dihydroxyacetone phosphate. (c) Dynamic relationship of M145 strain growth, Act synthesis and TAG pool. The amount of TAG at 20 h is normalized as one. The average value of multiple experiments (grey curves) is shown as a dark curve. (d) Intracellular metabolites of M145 strain that are significantly related to glucose consumption at 20 h to 48 h and Act synthesis at 72 h to 96 h. (e) Transcription profile of genes involved in fatty acid synthesis pathway in M145. The dark curve on the right shows an overall trend of transcription trend levels of related genes. (f) Transcription profile of genes involved in fatty acid degradation pathway in M145. The dark curve on the right shows an overall trend of transcription trend levels of related genes.

FIG. 3 analyzes the mechanism of high-yielding Streptomyces strains mobilizing intracellular TAG pool. (a)-(b): Labeling degrees of five Act compounds in M145 and HY01 strains. (a) Tracing of the metabolic flux from TAG pool to Act by using [U-¹³C] oleate. (b) Tracing of the metabolic flux from glucose to Act by using [U-¹³C] glucose. In Figs. (a) and (b), M and H represent M145 and HY01, respectively. (c)-(e): Comparison of the NADH/NAD⁺ ratio, ATP/ADP ratio and NADPH/NADP⁺ ratio in M145 and HY01 during the fermentation stationary phase. (f)-(h): Influence of addition of NADH and/or ATP on the activities of α-ketoglutarate dehydrogenase (α-KGDH), isocitrate dehydrogenase (IDH) and citrate synthase (CS) in vitro. The enzyme activity value of the M145 cell extracts without adding NADH and ATP is set as 100%. (i) Metabolic flux distribution at the node of acetyl-coenzyme A nodes in strains M145 and HY01. Glc: glucose; PYR: pyruvate; TAG pool: triacylglycerol pool; AcCoA: acetyl-coenzyme A; Act: actinomycin; TCA cycle: citric acid cycle. All results are given as the average and s.d. of three independent experiments.

FIG. 4 shows the identification of key proteins that affect the utilization of the intracellular TAG pool. (a) A brief illustration of metabolic pathway from intracellular TAG pool to polyketide synthesis. (b) Homology analysis among the 888 acyl-coenzyme A synthases in 125 strains of Streptomyces. (c) Analysis of transcription intensity of five conserved acyl-coenzyme A synthases in M145 strain. (d) Analysis of transcription temporal of sco6196 in M145 and HY01. (e) Effects of knockout (6196DM) and overexpression (61960E) of sco6196 on Act biosynthesis of Streptomyces coelicolor. (f) TLC assay of the remaining intracellular TAG pools in M145, 6196DM and 61960E during the stationary phase. (g) GC-MS assay of the remaining intracellular TAG pools in M145, 6196DM and 61960E during the stationary phase. Chromatographic peaks 1 to 15 are iC13:0, aiC13:0, iC14:0, C14:0, iC15:0, aiC15:0, C15:0, iC16:0, C16:1-Δ9, C16:0, iC17:0, aiC17:0, C17:0, C18:1-Δ9 and C18:0, respectively. Data shown in Figs. (c)-(e) are the average and s.d. of three independent experiments.

FIG. 5 shows improving the production of different polyketides through timing regulation of TAG decomposition (ddTAG strategy). (a) Schematic of ddTAG module construction strategy. (b) Determination of the optimal induction time and induction dose for Streptomyces coelicolor M145-DT containing the ddTAG module. (c) Changes in glucose consumption and Act production of M145 and M145-DT strains under fed-batch fermentation conditions over time. Feeding means supplementing glucose as carbon sources. (d) Relative productions of Act in M145 and M145-DT strains under batch and fed-batch fermentation conditions. (e) Fermentation production of Jedomycin B (JdB) produced by Streptomyces venezuelae ISP5230 and Sv-DT. (f) Fermentation production of oxytetracycline produced by Streptomyces rimosus M4018, M-DT, M2R and M2R-DT. (g) Abamectin B1a production of A56 and A56-DT in a 180-ton fermenter. Data in Figs. (c)-(f) are shown as the average and s.d. of three independent experiments. Data in Figs. (d)-(f) are analyzed by t test (p<0.05 is considered statistically significant, and the significance levels are ***p<0.001, **p<0.01 and *p<0.05).

### DETAILED DESCRIPTION

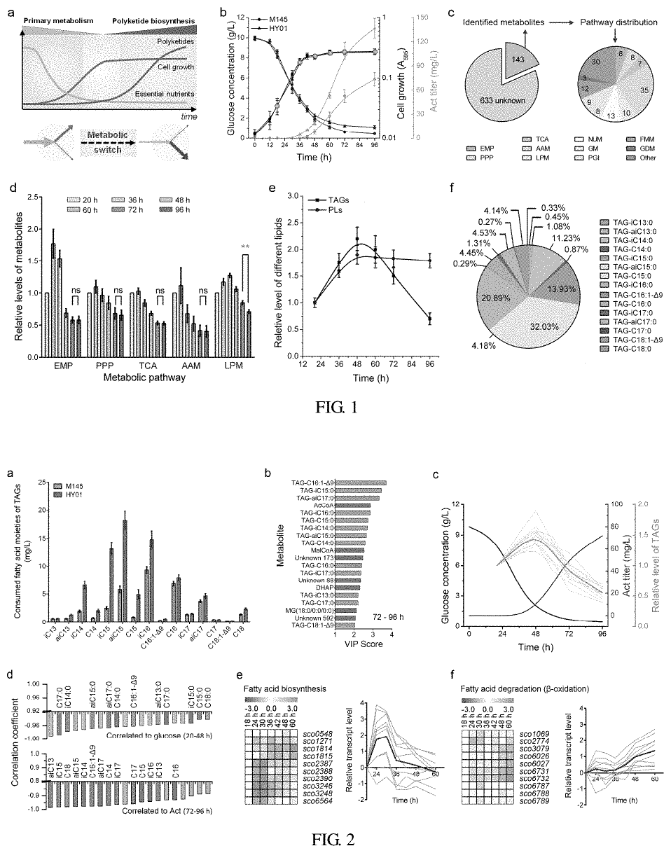

It is known in the art that, in a closed culture system, the growth of microorganisms is generally divided into a lag phase, an exponential phase (also known as a logarithmic growth phase), a stationary phase (wherein the specific growth rate of bacteria is zero), and a decay phase. In the exponential period, the medium is rich in nutrients and the number of microorganisms increases substantially. However, due to large consumption of nutrients in the exponential phase, remaining nutrients are not enough to support the continued mass reproduction of microorganisms. The population of microorganisms slows down during the stationary phase, a relatively stable number of individuals is maintained, and a large number of secondary metabolites are produced (FIG. 1a). In the present disclosure, it is proposed for the first time that the production of secondary metabolites can be promoted by enhancing a triacylglycerol decomposition during the stationary phase. The triacylglycerol may come from triacylglycerol accumulated by bacteria itself, or may come from a culture environment, such as soybean oil, palm oil, gutter oil, etc., added to a culture medium. The terms “primary metabolism” and “secondary metabolism” used herein have the meanings known in the art. Primary metabolism is a metabolism (such as energy metabolism, and synthesis of amino acid, protein, nucleic acid, etc.) that directly involves basic biological functions such as growth and development that exists throughout the growth cycle of a variety of organisms. Secondary metabolism (also known as secondary metabolite) is a metabolic reaction that occurs only during a specific growth phase of a specific species. Although secondary metabolism is not involved in normal growth, development and reproduction, lack of secondary metabolites does not result in immediate death of the organism, but it is generally considered to result in a reduced viability of organisms. Secondary metabolites are produced during the stationary phase of fermentation.

Polyketide compounds are secondary metabolites synthesized by bacteria, fungi, plants and animals. The biosynthetic pathway is mainly as follows: adding a two-carbon unit of malonyl-CoA to an extending carbon chain through clathenate condensation reaction. Polyketide compounds can be roughly divided into three categories: a type I polyketide compound (a macrolide typically catalyzed by a multimodular megasynthase), a type II polyketide compound (an aromatic molecule typically produced by an iterative reaction of resolvase) and a type III polyketide compound (a small aromatic molecule typically produced by fungal species). A polyketide compound involved in the present disclosure comprise various polyketide compounds that can be produced by the fermentation of Streptomyces, such as, but not limited to an actinomycin, a jedomycin, an abamectin, a milbemycin, an oxytetracycline, a nemadectin, etc.

As used herein, the term “triacylglycerol” is also called triglyceride (abbreviated as TG, TAG), which is an ester organic compound formed by esterification of one glycerol molecule and three fatty acid molecules. Its general formula is CH₂COOR—CHCOOR′—CH₂COOR″, wherein R, R′, R″ are the same, partly the same or different long alkyl chains. As a main component of oil, triacylglycerol can be decomposed into glycerol and fatty acids to provide energy for organisms. In the present disclosure, the triacylglycerol which provides carbon sources and reducing power for secondary metabolism can be any triglyceride present in Streptomyces cells, the fatty acid moieties of which can be saturated or unsaturated (monounsaturated or polyunsaturated) having a carbon number of, for example, 12-24. The fatty acid moieties of triacylglycerol in the present disclosure can be an even-numbered carbon fatty acid or an odd-numbered carbon fatty acid, such as a dodecanoic acid, a tridecanoic acid, a tetradecanoic acid, a pentadecanoic acid, a hexadecanoic acid, a heptadecanoic acid, an octadecanoic acid, a nonadecanic acid, an eicosanic acid, a behenic acid, a tetracosanoic acid, etc.

Metabolism of fatty acids in organisms means fatty acids are decomposed through β-oxidation into acetyl-coenzyme A, which then enters a tricarboxylic acid cycle for further oxidation. In this process, enzymes that catalyze an irreversible step comprise, for example, an acyl-coenzyme A synthase, an acyl-coenzyme A dehydrogenase, and an acyl-coenzyme A hydratase.

Streptomyces that can be regulated by the method in the present disclosure comprise any industrial Streptomyces of the genus Streptomyces, comprising but not limited to a Streptomyces coelicolor, a Streptomyces albus, a Streptomyces venezuelae, a Streptomyces lividans, a Streptomyces avermitilis, a Streptomyces rimosus and a Streptomyces bingchenggensis.

In Streptomyces coelicolor, acyl-coenzyme A synthetase can be SCO1330 (having a NCBI registration number of 1096753), SCO2131 (having a NCBI registration number of 1097565), SCO2444 (having a NCBI registration number of 1097878), SCO2561 (having a NCBI registration number of 1097995), SCO2720 (having a NCBI registration number of 1098154), SCO3436 (having a NCBI registration number of 1098873), SCO4006 (having a NCBI registration number of 1099442), SCO4503 (having a NCBI registration number of 1099943), SCO5983 (having a NCBI registration number of 1101425), SCO6196 (having a NCBI registration number of 1101637), SCO6552 (having a NCBI registration number of 1101991), SCO6790 (having a NCBI registration number of 1102229), SCO6968 (having a NCBI registration number of 1102406), SCO7244 (having a NCBI registration number of 1102682), SCO7329 (having a NCBI registration number of 1102767), and SCO4383 (having a NCBI registration number of 1099823). Acyl-coenzyme A dehydrogenase can be SCO1690 (having a NCBI registration number of 1097121), SCO2774 (having a NCBI registration number of 1098208), and SCO6787 (having a NCBI registration number of 1102226). Acyl-coenzyme A hydratase can be SCO4384 (having a NCBI registration number of 1099824) and SCO6732 (having a NCBI registration number of 1102171).

In Streptomyces albus, acyl-coenzyme A synthetase can be SLNWT_0050 (having a NCBI registration number of 749644876), SLNWT_0304 (having a NCBI registration number of 749645099), SLNWT_0327 (having a NCBI registration number of 749645115), SLNWT_0598 (having a NCBI registration number of 749645407), SLNWT_0621 (having a NCBI registration number of 749645437), SLNWT_3453 (having a NCBI registration number of 1154940117), SLNWT_4291 (having a NCBI registration number of 912432010), SLNWT_6199 (having a NCBI registration number of 749654619), and SLNWT_6951 (having a NCBI registration number of 749655284). Acyl-coenzyme A dehydrogenase can be SLNWT_4686 (having a NCBI registration number of 749652342). Acyl-coenzyme A hydratase can be SLNWT_0723 (having a NCBI registration number of 749645556), SLNWT_0850 (having a NCBI registration number of 749645763), SLNWT_4292 (having a NCBI registration number of 749651797), SLNWT_6769 (having a NCBI registration number of 749655095), and SLNWT_6771 (having a NCBI registration number of 749655096).

In Streptomyces venezuelae, acyl-coenzyme A synthetase can be SVEN_0294 (having a NCBI registration number of 504844398), SVEN_0876 (having a NCBI registration number of 504844980), SVEN_2231 (having a NCBI registration number of 1154133092), SVEN_3097 (having a NCBI registration number of 75396681), SVEN_4199 (having a NCBI registration number of 753966229), SVEN_6078 (having a NCBI registration number of 504850157), SVEN_6188 (having a NCBI registration number of 504850267), SVEN_6773 (having a NCBI registration number of 504850852), SVEN_6774 (having a NCBI registration number of 504850853), and SVEN_7224 (having a NCBI registration number of 753967331). Acyl-coenzyme A dehydrogenase can be SVEN_0520 (having a NCBI registration number of 504844624), and SVEN_1293 (having a NCBI registration number of 504845396). Acyl-coenzyme A hydratase can be SVEN_0030 (having a NCBI registration number of 504844136), SVEN_0204 (having a NCBI registration number of 1368970457), SVEN_0279 (having a NCBI registration number of 504844384), SVEN_1657 (having a NCBI registration number of 504845760), SVEN_4200 (having a NCBI registration number of 504848295), SVEN_5574 (having a NCBI registration number of 504849653), SVEN_5576 (having a NCBI registration number of 504849655), and SVEN_6413 (having a NCBI registration number of 504850492).

In Streptomyces lividans, acyl-coenzyme A synthetase can be SLIV_03075 (having a NCBI registration number of 490069726), SLIV_04410 (having a NCBI registration number of 490070002), SLIV_07155 (having a NCBI registration number of 490070554), SLIV_16515 (having a NCBI registration number of 490072425), SLIV_25480 (having a NCBI registration number of 490074187), and SLIV_36365 (having a NCBI registration number of 490076385). Acyl-coenzyme A dehydrogenase can be SLIV_29290 (having a NCBI registration number of 511095400). The acyl-coenzyme A hydratase can be SLIV_16510 (having a NCBI registration number of 490072424), and SLIV_36115 (having a NCBI registration number of 490076331).

In Streptomyces avermitilis, acyl-coenzyme A synthetase can be SAVERM_1258 (having a NCBI registration number of WP_010982696.1), SAVERM_1346 (having a NCBI registration number of WP_010982784.1), SAVERM_1603 (having a NCBI registration number of WP_010983042.1), SAVERM_2030 (having a NCBI registration number of WP_010983470.1), SAVERM_2279 (having a NCBI registration number of WP_010983718.1), SAVERM_377 (having a NCBI registration number of WP_010981813.1), SAVERM_3806 (having a NCBI registration number of WP_010985237.1), SAVERM_3864 (having a NCBI registration number of WP_010985295.1), SAVERM_5723 (having a NCBI registration number of WP_010987125.1), SAVERM_605 (having a NCBI registration number of WP_037651173.1), and SAVERM_6612 (having a NCBI registration number of WP_010988013.1). Acyl-coenzyme A dehydrogenase can be SAVERM_1381 (having a NCBI registration number of WP_010982819.1), SAVERM_5280 (having a NCBI registration number of WP_010986684.1), and SAVERM_6614 (having a NCBI registration number of WP_010988015.1). Acyl-coenzyme A hydratase can be SAVERM_1245 (having a NCBI registration number of WP_037646088.1), SAVERM_1680 (having a NCBI registration number of WP_010983119.1), SAVERM_3863 (having a NCBI registration number of WP_010985294.1), SAVERM_6203 (having a NCBI registration number of WP_010987604.1), SAVERM_717 (having a NCBI registration number of WP_010982155.1), and SAVERM_7216 (having a NCBI registration number of WP_010988611.1).

In Streptomyces rimosus, the NCBI registration numbers of acyl-coenzyme A synthetase are WP_053803359.1, ELQ77730.1, WP_033034442.1, KOT44666.1, and WP_033033106.1, respectively. The NCBI registration numbers of acyl-coenzyme A dehydrogenase are WP_125057199.1, WP_033031914.1, WP_030661846.1, WP_030634872.1, and WP_030370993.1, respectively. The NCBI registration numbers of acyl-coenzyme A hydratase are WP_030669923.1 and WP_125053679.1, respectively.

In Streptomyces bingchenggensis, acyl-coenzyme A synthetase can be SBI_00524 (having a NCBI registration number of WP_014173124.1), SBI_02958 (having a NCBI registration number of 503941562), SBI_03178 (having a NCBI registration number of 1154238763), SBI_04546 (having a NCBI registration number of 1154238845), SBI_04871 (having a NCBI registration number of 759782012), SBI_06310 (having a NCBI registration number of 503944887), SBI_07635 (having a NCBI registration number of 503946211), SBI_08381 (having a NCBI registration number of 503946955), SBI_08662 (having a NCBI registration number of 759784200), and SBI_09123 (having a NCBI registration number of 503947694). Acyl-coenzyme A dehydrogenase can be SBI_08383 (having a NCBI registration number of 503946957) and SBI_09842 (having a NCBI registration number of 759779066). Acyl-coenzyme A hydratase can be SBI_01088 (having a NCBI registration number of 503939694), SBI_01673 (having a NCBI registration number of 503940279), SBI_01731 (having a having a NCBI registration number of 503940337), SBI_02642 (having a NCBI registration number of 503941246), and SBI_04870 (having a NCBI registration number of 503943465).

In the present disclosure, it is also possible to promote β-oxidation pathway by expressing a protein having a corresponding function and having a homology of 70% or more, 80% or more, 85% or more, preferably 95% or more and 99% or more with the above-mentioned acyl-coenzyme A synthetase, acyl-coenzyme A dehydrogenase and acyl-coenzyme A hydratase in Streptomyces.

In some embodiments, the triacylglycerol decomposition can be enhanced by overexpressing endogenous esterase or expressing exogenous esterase in Streptomyces, thereby promoting the synthesis of polyketide compounds. The esterase can be an intracellular or extracellular esterase (EC: 3.1.1.3) that catalyzes the degradation of intracellular or extracellular triacylglycerol to produce fatty acids. The esterase can be selected from, for example, a Streptomyces coelicolor esterase selected from SCO0713 (NP_625018.1), SCO1265 (NP_625552.1), SCO1735 (NP_626008.1), SCO3219 (NP_627433.1), SCO4368 (NP_628538.1), SCO4746 (NP_628904.1), SCO4799 (NP_628956.1), SCO6966 (NP_631032.1) and SCO7131 (NP_631192.1); a Streptomyces bingchenggensis esterase selected from SBI_00115 (WP_014172715.1), SBI_00631 (WP_014173231.1), SBI_01149 (WP_014173749.1), SBI_01728 (WP_014174328.1); a Streptomyces avermitilis esterase selected from SAVERM_RS02860 (WP_010981907.1), SAVERM_RS04345 (WP_010036168.1), SAVERM_RS04550 (WP_107083239.1) and SAVERM_RS23405 (WP_010985956.1); a Streptomyces albus esterase selected from SLNWT_RS18180 (WP_078845043.1), SLNWT_RS12910 (WP_040249758.1), SLNWT_RS12900 (WP_040249752.1); and a Bacillus subtilis esterase selected from BSU_08350 (NP_388716.1), BSU_24510 (NP_390331.1) and BSU_21740 (NP_390057.1). The esterase may also be from other microbials, animals or plants. In the present disclosure, β-oxidation pathway can be promoted by expressing a protein having an esterase activity and having 70% or more, 80% or more, 85% or more, preferably 95% or more, 99% or more homology with the above-mentioned esterase in Streptomyces.

As used to describe a polypeptide or a protein, the term “homology” as used herein means that at least 70%, usually about 75%-99%, and more preferably at least about 98%-99% of the amino acids in two polypeptides are identical when performing an optimal alignment (for example, BLAST with default parameters for alignment is used).

It is known in the art that the coding sequence can be operably linked to downstream of an inducible promoter to dynamically regulate the expression of a certain gene by means of the inducible promoter, and an inducer can be added at a required time to dynamically turn on or turn off the expression of this gene. An expression cassette containing this inducible promoter and coding sequence can be integrated into the genome or be present on an exogenous plasmid. In the present disclosure, the production of polyketide compounds (such as abamectin) is increased by turning on or enhancing the expression of genes related to the TAG degradation pathway during the stationary phase. Inducible promoters commonly used in various Streptomyces have been characterized in the art, such as those described in Horbal et al., 2014, Appl Microbiol Biotechnol, 98:8641-8655 and Wang et al. 2016, ACS Synth Biol, 5:765-773.

As used herein, all the terms “increase”, “promote/improve”, “enhance”, “activate” or “overexpress” generally refer to increase by a statistically significant amount. For the avoidance of doubt, the terms “increase”, “promote/improve”, “enhance”, “activate” or “overexpress” usually means an increase of at least 10% compared with strains without genetic modification, for example, an increase of at least about 20%, or at least about 30%, or at least about 40%, or at least about 50%, or at least about 60%, or at least about 70%, or at least about 80%, or at least about 90%, or up to and including an increase of 100%, or any increase between 10%-100% compared with strains without genetic modification; or at least about 2-fold, or at least about 3-fold, or at least about 4-fold, or at least about 5-fold, or at least about 10-fold, or any increase between 2-fold to 10-fold compared with strains without genetic modification.

Implementations of each aspect described herein can be illustrated by the following numbered paragraphs:

1. A method for improving the production of a polyketide compound in a Streptomyces, comprising a step of strengthening a triacylglycerol decomposition pathway in a Streptomyces, and preferably the Streptomyces during a stationary phase.

2. The method of paragraph 1, wherein the triacylglycerol decomposition pathway is a β-oxidation pathway.

3. The method of paragraph 1 or 2, wherein the polyketide compound is selected from the group consisting of a type I polyketone compound, a type II polyketone compound, and a type III polyketone compound.

4. The method of paragraph 3, wherein the polyketide is selected from the group consisting of actinomycin, jadomycin, avermectin, milbemycin, oxytetracycline and nemadectin.

5. The method of any one of paragraphs 1-4, wherein a fatty acid moiety of the triacylglycerol is a fatty acid having a carbon number of 12-24.

6. The method of any one of paragraphs 1-5, wherein the Streptomyces is selected from the group consisting of a Streptomyces coelicolor, a Streptomyces albus, a Streptomyces venezuelae, Streptomyces lividans, a Streptomyces avermitilis, a Streptomyces rimosus, a Streptomyces hygroscopicus, a Streptomyces cyaneogriseus, and a Streptomyces bingchenggensis.

7. The method of any one of paragraphs 1-6, wherein the triacylglycerol decomposition pathway is strengthened by enhancing an expression level and/or activity of at least one enzyme in the Streptomyces that catalyzes an irreversible reaction of the β-oxidation pathway.

8. The method of paragraph 7, wherein the enzyme that catalyzes an irreversible reaction of the β-oxidation pathway is selected from the group consisting of an acyl-coenzyme A synthetase, an acyl-coenzyme A dehydrogenase, an acyl-coenzyme A hydratase, and any combination thereof.

9. The method of paragraph 8, wherein the Streptomyces is a Streptomyces coelicolor, the acyl-coenzyme A synthetase is selected from the group consisting of SCO1330, SCO2131, SCO2444, SCO2561, SCO2720, SCO3436, SCO4006, SCO4503, SCO5983, SCO6196, SCO6552, SCO6790, SCO6968, SCO7244, SCO7329, SCO4383, and any combination thereof; the acyl-coenzyme A dehydrogenase is selected from the group consisting of SCO1690, SCO2774, SCO6787, and any combination thereof; and the acyl-coenzyme A hydratase is selected from SCO4384 and/or SCO6732.

10. The method of paragraph 8, wherein the Streptomyces is a Streptomyces albus, the acyl coenzyme A synthetase is selected from the group consisting of SLNWT_0050, SLNWT_0304, SLNWT_0327, SLNWT_0598, SLNWT_0621, SLNWT_3453, SLNWT_4291, SLNWT_6199, SLNWT_6951, and any combination thereof; the acyl-coenzyme A dehydrogenase is SLNWT_4686; and the acyl-coenzyme A hydratase is selected from the group consisting of SLNWT_0723, SLNWT_0850, SLNWT_4292, SLNWT_6769, SLNWT_6771, and any combination thereof.

11. The method of paragraph 8, wherein the Streptomyces is a Streptomyces venezuelae, the acyl-coenzyme A synthetase is selected from the group consisting of SVEN_0294, SVEN_0876, SVEN_2231, SVEN_3097, SVEN_4199, SVEN_6078, SVEN_6188, SVEN_6773, SVEN_6774, SVEN_7224, and any combination thereof; the acyl-coenzyme A dehydrogenase is selected from SVEN_0520 and/or SVEN_1293; and the acyl-coenzyme A hydratase is selected from the group consisting of SVEN_0030, SVEN_0204, SVEN_0279, SVEN_1657, SVEN_4200, SVEN_5574, SVEN_5576, SVEN_6413, and any combination thereof.

12. The method of paragraph 8, wherein the Streptomyces is a Streptomyces lividans, the acyl-coenzyme A synthetase is selected from the group consisting of SLIV_03075, SLIV_04410, SLIV_07155, SLIV_16515, SLIV_25480, SLIV_36365, and any combination thereof; the acyl-coenzyme A dehydrogenase is SLIV_29290; and the acyl-coenzyme A hydratase is selected from SLIV_16510 and/or SLIV_36115.

13. The method of paragraph 8, wherein the Streptomyces is a Streptomyces avermitilis, the acyl-coenzyme A synthetase is selected from the group consisting of SAVERM_1258, SAVERM_1346, SAVERM_1603, SAVERM_2030, SAVERM_2279, SAVERM_377, SAVERM_3806, SAVERM_3864, SAVERM_5723, SAVERM_605 and SAVERM_6612; the acyl-coenzyme A dehydrogenase is selected from the group consisting of SAVERM_1381, SAVERM_5280, SAVERM_6614, and any combination thereof; and the acyl-coenzyme A hydratase is selected from SAVERM_1245, SAVERM_1680, SAVERM_3863, SAVERM_6203, SAVERM_717 and/or SAVERM_7216.

14. The method of paragraph 8, wherein the Streptomyces is a Streptomyces rimosus, the acyl-coenzyme A synthetase is selected from the group consisting of an acyl-coenzyme A synthetase having a NCBI registration number of WP_053803359.1, ELQ77730.1, WP_033034442.1, KOT44666.1, WP_033033106.1, and any combination thereof; the acyl-coenzyme A dehydrogenase is selected from the group consisting of an acyl-coenzyme A synthetase having a NCBI registration number of WP_125057199.1, WP_033031914.1, WP_030661846.1, WP_030634872.1, WP_030370993.1, and any combination thereof; and the acyl-coenzyme A hydratase is selected from an acyl-coenzyme A hydratase having a NCBI registration number of WP_030669923.1 and/or WP_125053679.1.

15. The method of paragraph 8, wherein the Streptomyces is a Streptomyces bingchenggensis, the acyl-coenzyme A synthetase is selected from the group consisting of SBI_00524, SBI_02958, SBI_03178, SBI_04546, SBI_04871, SBI_06310, SBI_07635, SBI_08381, SBI_08662, SBI_09123, and any combination thereof; the acyl-coenzyme A dehydrogenase is selected from SBI_08383 and/or SBI_09842; and the acyl-coenzyme A hydratase is selected from the group consisting of SBI_01088, SBI_01673, SBI_01731, SBI_02642, SBI_04870, and any combination thereof.

16. The method of paragraph 6, wherein the triacylglycerol decomposition pathway is strengthened by enhancing an expression level and/or activity of an esterase in the Streptomyces.

17. The method of paragraph 16, wherein the esterase is selected from the group consisting of the following proteins: a Streptomyces coelicolor esterase selected from SCO0713 (NP_625018.1), SCO1265 (NP_625552.1), SCO1735 (NP_626008.1), SCO3219 (NP_627433.1), SCO4368 (NP_628538.1), SCO4746 (NP_628904.1), SCO4799 (NP_628956.1), SCO6966 (NP_631032.1) and SCO7131 (NP_631192.1); a Streptomyces bingchenggensis esterase selected from SBI_00115 (WP_014172715.1), SBI_00631 (WP_014173231.1), SBI_01149 (WP_014173749.1) and SBI_01728 (WP_014174328.1); a Streptomyces avermitilis esterase selected from SAVERM_RS02860 (WP_010981907.1), SAVERM_RS04345 (WP_010036168.1), SAVERM_RS04550 (WP_107083239.1), and SAVERM_RS23405(WP_010985956.1); a Streptomyces albus esterase selected from SLNWT_RS18180 (WP_078845043.1), SLNWT_RS12910 (WP_040249758.1) and SLNWT_RS12900 (WP_040249752.1); and a Bacillus subtilis esterase selected from BSU_08350 (NP_388716.1), BSU_24510 (NP_390331.1) and BSU_21740 (NP_390057.1).

18. The method of any one of paragraphs 8-17, wherein the enzyme is operably linked to downstream of an inducible promoter and induced during the stationary phase.

19. The method of any one of paragraphs 1-18, wherein the triacylglycerol decomposition pathway in the Streptomyces is strengthened during the stationary phase by inhibiting a carbon metabolism flow from an acetyl-coenzyme A to a tricarboxylic acid cycle.

20. The method of any one of paragraphs 1-19, further comprising increasing a NADH/NAD+ ratio in the Streptomyces.

21. The method of paragraph 20, wherein the NADH/NAD⁺ ratio is increased by adding NADH and/or ATP to a medium.

22. A method for switching a primary metabolism to a secondary metabolism in a Streptomyces, comprising strengthening a triacylglycerol decomposition pathway in the Streptomyces.

23. The method of paragraph 22, wherein the triacylglycerol decomposition pathway is a β-oxidation pathway.

24. The method of paragraph 22 or 23, wherein a fatty acid moiety of the triacylglycerol is a fatty acid having a carbon number of 12-24.

25. The method of any one of paragraphs 22-24, wherein the Streptomyces is selected from the group consisting of a Streptomyces coelicolor, a Streptomyces albus, a Streptomyces venezuelae, a Streptomyces lividans, a Streptomyces avermitilis, a Streptomyces rimosus, Streptomyces hygroscopicus, a Streptomyces cyaneogriseus, and a Streptomyces bingchenggensis.

26. The method of any one of paragraphs 22-25, wherein the triacylglycerol decomposition pathway is strengthened by enhancing an expression level and/or activity of at least one enzyme in the Streptomyces that catalyzes an irreversible reaction of the β-oxidation pathway.

27. The method of paragraph 26, wherein the enzyme that catalyzes an irreversible reaction of the β-oxidation pathway is selected from the group consisting of an acyl-coenzyme A synthetase, an acyl-coenzyme A dehydrogenase, and an acyl-coenzyme A hydratase.

28. The method of paragraph 27, wherein the Streptomyces is a Streptomyces coelicolor, the acyl-coenzyme A synthetase is selected from the group consisting of SCO1330, SCO2131, SCO2444, SCO2561, SCO2720, SCO3436, SCO4006, SCO4503, SCO5983, SCO6196, SCO6552, SCO6790, SCO6968, SCO7244, SCO7329, SCO4383, and any combination thereof; the acyl-coenzyme A dehydrogenase is selected from the group consisting of SCO1690, SCO2774, SCO6787, and any combination thereof; and the acyl-coenzyme A hydratase is selected from SCO4384 and/or SCO6732.

29. The method of paragraph 27, wherein the Streptomyces is a Streptomyces albus, the acyl-coenzyme A synthetase is selected from the group consisting of SLNWT_0050, SLNWT_0304, SLNWT_0327, SLNWT_0598, SLNWT_0621, SLNWT_3453, SLNWT_4291, SLNWT_6199, SLNWT_6951, and any combination thereof; the acyl-coenzyme A dehydrogenase is SLNWT_4686; and the acyl-coenzyme A hydratase is selected from the group consisting of SLNWT_0723, SLNWT_0850, SLNWT_4292, SLNWT_6769, SLNWT_6771, and any combination thereof.

30. The method of paragraph 27, wherein the Streptomyces is a Streptomyces venezuelae, the acyl-coenzyme A synthetase is selected from the group consisting of SVEN_0294, SVEN_0876, SVEN_2231, SVEN_3097, SVEN_4199, SVEN_6078, SVEN_6188, SVEN_6773, SVEN_6774, SVEN_7224, and any combination thereof; the acyl-coenzyme A dehydrogenase is selected from SVEN_0520 and/or SVEN_1293; and the acyl-coenzyme A hydratase is selected from the group consisting of SVEN_0030, SVEN_0204, SVEN_0279, SVEN_1657, SVEN_4200, SVEN_5574, SVEN_5576, SVEN_6413, and any combination thereof.

31. The method of paragraph 27, wherein the Streptomyces is a Streptomyces lividans, the acyl-coenzyme A synthetase is selected from the group consisting of SLIV_03075, SLIV_04410, SLIV_07155, SLIV_16515, SLIV_25480, SLIV_36365, and any combination thereof; the acyl-coenzyme A dehydrogenase is SLIV_29290; and the acyl-coenzyme A hydratase is selected from SLIV_16510 and/or SLIV_36115.

32. The method of paragraph 27, wherein the Streptomyces is a Streptomyces avermitilis, the acyl-coenzyme A synthetase is selected from the group consisting of SAVERM_1258, SAVERM_1346, SAVERM_1603, SAVERM_2030, SAVERM_2279, SAVERM_377, SAVERM_3806, SAVERM_3864, SAVERM_5723, SAVERM_605 and SAVERM_6612; the acyl-coenzyme A dehydrogenase is selected from the group consisting of SAVERM_1381, SAVERM_5280, SAVERM_6614, and any combination thereof; and the acyl-coenzyme A hydratase is selected from SAVERM_1245, SAVERM_1680, SAVERM_3863, SAVERM_6203, SAVERM_717 and/or SAVERM_7216.

33. The method of paragraph 27, wherein the Streptomyces is Streptomyces rimosus, the acyl-coenzyme A synthetase is selected from the group consisting of an acyl-coenzyme A synthetase having a NCBI registration number of WP_053803359.1, ELQ77730.1, WP_033034442.1, KOT44666.1, WP_033033106.1, and any combination thereof; the acyl-coenzyme A dehydrogenase is selected from the group consisting of an acyl coenzyme A synthetase having a NCBI registration number of WP_125057199.1, WP_033031914.1, WP_030661846.1, WP_030634872.1, WP_030370993.1, and any combination thereof; and the acyl-coenzyme A hydratase is selected from an acyl-coenzyme A hydratase having a NCBI registration number of WP_030669923.1 and/or WP_125053679.1.

34. The method of paragraph 27, wherein the Streptomyces is a Streptomyces bingchenggensis, the acyl-coenzyme A synthetase is selected from the group consisting of SBI_00524, SBI_02958, SBI_03178, SBI_04546, SBI_04871, SBI_06310, SBI_07635, SBI_08381, SBI_08662, SBI_09123, and any combination thereof; the acyl-coenzyme A dehydrogenase is selected from SBI_08383 and/or SBI_09842; and the acyl-coenzyme A hydratase is selected from the group consisting of SBI_01088, SBI_01673, SBI_01731, SBI_02642, SBI_04870, and any combination thereof.

35. The method of paragraphs 22-25, wherein the triacylglycerol decomposition pathway is strengthened by enhancing an expression level and/or activity of an esterase in the Streptomyces.

36. The method of paragraph 35, wherein the esterase is selected from the group consisting of the following proteins: a Streptomyces coelicolor esterase selected from SCO0713 (NP_625018.1), SCO1265 (NP_625552.1), SCO1735 (NP_626008.1), SCO3219 (NP_627433.1), SCO4368 (NP_628538.1), SCO4746 (NP_628904.1), SCO4799 (NP_628956.1), SCO6966 (NP_631032.1) and SCO7131 (NP_631192.1); a Streptomyces bingchenggensis esterase selected from SBI_00115 (WP_014172715.1), SBI_00631 (WP_014173231.1), SBI_01149 (WP_014173749.1) and SBI_01728 (WP_014174328.1); a Streptomyces avermitilis esterase selected from SAVERM_RS02860 (WP_010981907.1), SAVERM_RS04345 (WP_010036168.1), SAVERM_RS04550 (WP_107083239.1), and SAVERM_RS23405(WP_010985956.1); a Streptomyces albus esterase selected from SLNWT_RS18180 (WP_078845043.1), SLNWT_RS12910 (WP_040249758.1) and SLNWT_RS12900 (WP_040249752.1); and a Bacillus subtilis esterase selected from BSU_08350 (NP_388716.1), BSU_24510 (NP_390331.1) and BSU_21740 (NP_390057.1).

37. The method of any one of paragraphs 27-36, wherein the enzyme is operably linked to downstream of an inducible promoter, and switching from a primary metabolism to a secondary metabolism is achieved by inducing expression.

38. A Streptomyces for producing a polyketide compound by fermentation, wherein an expression level and/or activity of at least one enzyme in the Streptomyces that catalyzes an irreversible reaction of a β-oxidation pathway is enhanced compared with an original strain.

39. The Streptomyces of paragraph 38, wherein at least one enzyme that catalyzes an irreversible reaction of the β-oxidation pathway is provided downstream of an inducible promoter.

40. The Streptomyces of paragraph 39, wherein the enzyme that catalyzes an irreversible reaction of the β-oxidation pathway is selected from the group consisting of an acyl-coenzyme A synthetase, an acyl-coenzyme A dehydrogenase, and an acyl-coenzyme A hydratase.

41. The Streptomyces of paragraph 40, wherein the Streptomyces is a Streptomyces coelicolor, and the acyl-coenzyme A synthetase is selected from the group consisting of SCO1330, SCO2131, SCO2444, SCO2561, SCO2720, SCO3436, SCO4006, SCO4503, SCO5983, SCO6196, SCO6552, SCO6790, SCO6968, SCO7244, SCO7329, SCO4383, and any combination thereof; the acyl-coenzyme A dehydrogenase is selected from the group consisting of SCO1690, SCO2774, SCO6787, and any combination thereof; and the acyl-coenzyme A hydratase is selected from SCO4384 and/or SCO6732.

42. The Streptomyces of paragraph 40, wherein the Streptomyces is a Streptomyces albus, the acyl-coenzyme A synthetase is selected from the group consisting of SLNWT_0050, SLNWT_0304, SLNWT_0327, SLNWT_0598, SLNWT_0621, SLNWT_3453, SLNWT_4291, SLNWT_6199, SLNWT_6951, and any combination thereof; the acyl-coenzyme A dehydrogenase is SLNWT_4686; and the acyl-coenzyme A hydratase is selected from the group consisting of SLNWT_0723, SLNWT_0850, SLNWT_4292, SLNWT_6769, SLNWT_6771, and any combination thereof.

43. The Streptomyces of paragraph 40, wherein the Streptomyces is a Streptomyces venezuelae, the acyl-coenzyme A synthetase is selected from the group consisting of SVEN_0294, SVEN_0876, SVEN_2231, SVEN_3097, SVEN_4199, SVEN_6078, SVEN_6188, SVEN_6773, SVEN_6774, SVEN_7224, and any combination thereof; the acyl-coenzyme A dehydrogenase is selected from SVEN_0520 and/or SVEN_1293; and the acyl-coenzyme A hydratase is selected from the group consisting of SVEN_0030, SVEN_0204, SVEN_0279, SVEN_1657, SVEN_4200, SVEN_5574, SVEN_5576, SVEN_6413, and any combination thereof.

44. The Streptomyces of paragraph 40, wherein the Streptomyces is a Streptomyces lividans, and the acyl-coenzyme A synthetase is selected from the group consisting of SLIV_03075, SLIV_04410, SLIV_07155, SLIV_16515, SLIV_25480, SLIV_36365, and any combination thereof; the acyl-coenzyme A dehydrogenase is SLIV_29290; and the acyl-coenzyme A hydratase is selected from SLIV_16510 and/or SLIV_36115.

45. The Streptomyces of paragraph 40, wherein the Streptomyces is a Streptomyces avermitilis, and the acyl-coenzyme A synthetase is selected from the group consisting of SAVERM_1258, SAVERM_1346, SAVERM_1603, SAVERM_2030, SAVERM_2279, SAVERM_377, SAVERM_3806, SAVERM_3864, SAVERM_5723, SAVERM_605 and SAVERM_6612; the acyl-coenzyme A dehydrogenase is selected from the group consisting of SAVERM_1381, SAVERM_5280, SAVERM_6614, and any combination thereof; and the acyl-coenzyme A hydratase is selected from SAVERM_1245, SAVERM_1680, SAVERM_3863, SAVERM_6203, SAVERM_717 and/or SAVERM_7216.

46. The Streptomyces of paragraph 40, wherein the Streptomyces is a Streptomyces rimosus, and the acyl-coenzyme A synthetase is selected from the group consisting of an acyl-coenzyme A synthetase having a NCBI registration number of WP_053803359.1, ELQ77730.1, WP_033034442.1, KOT44666.1, WP_033033106.1, and any combination thereof; the acyl-coenzyme A dehydrogenase is selected from the group consisting of an acyl-coenzyme A synthetase having a NCBI registration number of WP_125057199.1, WP_033031914.1, WP_030661846.1, WP_030634872.1, WP_030370993.1, and any combination thereof; and the acyl-coenzyme A hydratase is selected from an acyl-coenzyme A hydratase having a NCBI registration number of WP_030669923.1 and/or WP_125053679.1.

47. The Streptomyces of paragraph 40, wherein the Streptomyces is a Streptomyces bingchenggensis, and the acyl-coenzyme A synthetase is selected from the group consisting of SBI_00524, SBI_02958, SBI_03178, SBI_04546, SBI_04871, SBI_06310, SBI_07635, SBI_08381, SBI_08662, SBI_09123, and any combination thereof; the acyl-coenzyme A dehydrogenase is selected from SBI_08383 and/or SBI_09842; and the acyl-coenzyme A hydratase is selected from the group consisting of SBI_01088, SBI_01673, SBI_01731, SBI_02642, SBI_04870, and any combination thereof.

48. The Streptomyces of any one of paragraphs 38-47, wherein the polyketide compound is selected from the group consisting of a type I polyketone compound, a type II polyketone compound, and a type III polyketone compound.

49. The Streptomyces of paragraph 48, wherein the polyketide is selected from the group consisting of actinomycin, jadomycin, avermectin, milbemycin, oxytetracycline and nemadectin.

50. The Streptomyces of any one of paragraphs 38-49, wherein a fatty acid moiety of the triacylglycerol is a fatty acid having a carbon number of 12-24.

51. The Streptomyces of any one of paragraphs 38-50, wherein the Streptomyces is selected from the group consisting of a Streptomyces coelicolor, a Streptomyces albus, a Streptomyces venezuelae, a Streptomyces lividans, a Streptomyces avermitilis, a Streptomyces rimosus, a Streptomyces hygroscopicus, a Streptomyces cyaneogriseus, and a Streptomyces bingchenggensis.

52. Use of the Streptomyces of any one of paragraphs 38-51 in the production of a polyketide compound by fermentation.

### Embodiments

Streptomyces coelicolor M145 used in the embodiments are purchased from ATCC (ATCC BAA-471). HY01 is preserved in the Institute of Microbiology, Chinese Academy of Sciences. Streptomyces venezuelae ISP5230 is purchased from ATCC (ATCC 10712). Streptomyces avermitilis A56 is as described in the following documents: Zhuo, Y. etc., Reverse biological engineering of hrdB to enhance the production of avermectins in an industrial strain of Streptomyces avermitilis. Proc Natl Acad Sci 107, 11250, USA (2010); and J. et al., Interrogation of Streptomyces avermitilis for efficient production of avermectins. Synth Syst Biotechnol 1, 7-16 (2016). Streptomyces rimosus M4018 and its high-yielding engineered strain M2R (i.e., strain M4018::2SFotrR) are from the following document: Yin, S., etc., Identification of a cluster-situated activator of oxytetracycline biosynthesis and management of its expression for improved oxytetracycline production in Streptomyces rimosus. Microb Cell Fact 14, 46 (2015). Unless otherwise specified, the reagents used in the present disclosure are purchased from Aladdin or Sigma. For media and buffers used in the experiments, please refer to appendix 2 of the second volume of “Molecular Cloning: A Laboratory Manual” (Third edition, Science Press, 2002). For the cultivation and molecular manipulation of Streptomyces, please refer to “Practical Streptomyces Genetics” (John⋅Innice Foundation, Norwich, U K, 2000).

Studies have shown that when Streptomyces are cultured on a solid medium, mycelia begin to die during a late stationary phase and release nutrients for secondary metabolite and morphological differentiation (Rigali, S., etc., EMBO reports 9, 670-675 (2008)). However, this phenomenon is not observed in Streptomyces cultured in liquid medium (van Dissel, D. et al., Adv Appl Microbiol 89, 1-45 (2014)). In addition, previous studies have shown that in a variety of liquid media, the intracellular concentration of many metabolites (such as intermediates of central carbon metabolic intermediates) involved in primary metabolism during the stationary phase, the regulatory levels of the corresponding genes, and activity of the enzymes that regulate these pathways decrease significantly (Nieselt, K., etc., BMC Genomics 11, 10 (2010); Wentzel, A., etc., Metabolites 2, 178-194 (2012); Jankevics, A., etc., PROTEOMICS 11, 4622-4631 (2011); Huang, J., etc., Genes Dev 15, 3183-3192 (2001); D'Huys, P J, etc., J Biotechnol 161, 1-13 (2012)). Theoretically, primary metabolic pathway should provide energy and material sources for the secondary metabolic pathway including biosynthesis of polyketide compounds. In fact, however, the primary metabolic pathway is nearly closed during the stationary phase (FIG. 1a). In this regard, the critical junction between primary metabolism and biosynthesis of polyketide compounds remains unknown. Due to the fact that materials and energies of the secondary metabolic process during the stationary phase are not from primary metabolic pathway, the inventors hypothesize that there may be some intracellular metabolite pool connecting the primary and secondary metabolism of Streptomyces. This metabolite pool provides precursors and energy sources for the biosynthesis of polyketide compounds. A model engineered strain Streptomyces coelicolor as an experimental object is used, and systematically study its time-course comparative metabolome and transcriptome, so as to reveal the key metabolic switch that dynamically controls the growth phase. By comparing the metabolome of wild-strain M145 with actinomycin high-yielding strain HY01, it is found that intracellular triacylglycerol (TAG) is a major intracellular carbon source in the biosynthetic pathway of polyketide compounds during the stationary phase. TAG is accumulated during primary metabolism and is consumed during polyketide synthesis. Dynamic regulation of accumulation/decomposition of TAG compounds acts as a switch in the process of switching from primary metabolism to biosynthesis of polyketide compounds. Our research also shows that the main mechanism for the closure of primary metabolism during the stationary phase is that TAG decomposition increases the NADH/NAD⁺ ratio, thereby inhibiting the enzymes in the TCA cycle, so that the metabolic flux entering the TCA cycle via acetyl-coenzyme A is reduced. These results indicate that the TAG pool is not only a main intracellular carbon source in secondary metabolism, but also a regulator of the carbon metabolic flux in the biosynthetic pathway of polyketide compounds. Therefore, it is proposed that the production of polyketide compounds can be increased by timing regulation of TAG decomposition (e.g., enhancing TAG decomposition by inducing acyl-coenzyme A synthase during secondary metabolism).

TAG pool is an intracellular carbon source during secondary metabolism: The batch fermentation of Streptomyces coelicolor wild-strain M145 and Actinopurin (Act) high-yielding strain HY01 show the following trends: Act is synthesized when the growth slows down, phosphorus source is limited, and glucose is about to be depleted. Compared with M145, HY01 can consume less glucose and synthesize more Act (FIG. 1b). In order to study the specific mechanism of this difference, we conduct a time-course comparative analysis of the metabolome of HY01 and M145. The culture medium is collected, and the cells are enriched at six different time points (20 h, 36 h, 48 h, 60 h, 72 h and 96 h) during the lag phase, the exponential phase and the stationary phase of liquid culture of HY01 and M145, and intracellular metabolites of the cells are analyzed by gas chromatography-mass spectrometry (GC-MS) method. A total of 776 metabolites with unique retention time are found in all samples. Comparing these metabolites with the National Institute of Standards and Technology (NIST 8.0) Mass Spectrometry Library and Fiehn Metabolism Library (Smart, K F, etc., Nat. protoc. 5, 1709-1729 (2010)). A total of 143 metabolites are identified (Table 1), which is involved almost all pathways related to primary metabolism (FIG. 1c). For M145, these metabolites show different trends over time: the metabolites related to primary metabolic pathways, including glycolysis (EMP), pentose phosphate pathway (PPP), tricarboxylic acid cycle (TCA) and amino acid metabolism (AAM), accumulate during the exponential phase (12-36 h), then decrease rapidly (36-72 h) and remain relatively constant throughout the stationary phase (72-96 h). However, the maximum concentration of the lipid metabolism pathway (LPM) appears at 48h, which is significantly later than other primary metabolites (36 h), and declines continuously during the late stationary phase (FIG. 1d). The time-course comparative metabolome analysis of HY01 shows a similar trend. Based on these results, it is hypothesized that the LPM pathway related to free fatty acids (FFA) and monoacylglycerols (MAG) may be the key to switching from primary metabolism to polyketide synthesis.

HA and MAG are the intermediates of cell lipid metabolism in Streptomyces, which mainly comprise phospholipids (PL) and triacylglycerols (TAG) (Olukoshi, E. R. & Packter, N. M., Microbiology 140 (Pt 4), 931-943 (1994)); Shim, M.-s., etc., Biotechnology Letters 19, 221-224 (1997)). As PL and TAG cannot be detected using GC-MS, thin-layer chromatography (TLC) is used to determine the amount of FFA and MAG at 20 h, 36 h, 48 h, 60 h, 72 h and 96 h of fermentation, so as to analyze the dynamic characteristics of PL and TAG As shown in FIG. 1e, TAG and PL gradually accumulate up to 48 h. Thereafter, the amount of PL remains relatively constant, whereas the amount of TAG decreases significantly. Further analysis of the fatty acid moieties obtained from TAG decomposition using GC-MS shows that for the 15 analyzed fatty acid moieties (FIG. 1f), the time-course change characteristics are the same as those of the TAG time-course change characteristics measured by TLC: decreasing continuously during the stationary phase (FIG. 1e). Compared with M145, more TAG is decomposed in HY01. Meanwhile, the stable level of other triacylglycerol metabolism intermediates, such as glycerol, 3-phosphoglycerate, dihydroxyacetone phosphate and glycerol 3-phosphate in HY01, is higher than that of M145. These data indicate that TAG pool may serve as an intracellular carbon pool, providing precursors when Act biosynthesis is significantly enhanced during the stationary phase, and external carbon source is insufficient.

Furthermore, changes in TAG pool during the stationary phase (48-96h) of M145 and HY01 strains are analyzed, and the fatty acid moieties obtained by TAG decomposition is quantitatively analyzed by GC-MS. The results show that the consumption of TAG pool in HY01 is significantly higher than the consumption of TAG pool in M145 (FIG. 2a). Supervised partial least squares discrimination analysis (PLS-DA) is performed on data of 776 intracellular metabolites obtained by GC-MS analysis, two coenzyme A precursors (acetyl-coenzyme A and malonyl-coenzyme A), fatty acid moieties of 16 TAGs, and contents of Act and glucose. The results show that during the whole stationary phase (60-96h), data of M145 and HY01 are divided into different clusters, indicating that concentrations of some metabolites vary greatly between the two strains, which may be the main reason for the difference in Act. The variable importance of projection (VIP) scores of each metabolite are ranked, which reflects the importance of the metabolite to Act production. The results show that among the 16 fatty acid fractions analyzed, 13 have major contributions to Act production (VIP score>1.5, 55 metabolites in total). Five of the ten metabolites that contribute the most to the Act production during the stationary phase are TAG fatty acid moieties. In view of the two strains, greatest difference in Act production appears during the late stationary phase (72-96h) (FIG. 1b), and the VIP scores during the late stationary phase (72-96h) are again ranked. As shown in FIG. 2b, the top ten ranked metabolites are all fatty acid moieties of TAG, except for two fatty acid metabolic precursors (acetyl-coenzyme A and malonyl-coenzyme A). In addition, the VIP scores of the fatty acid moieties of TAG during the stationary phase increase over time, indicating that the TAG pool has an increased influence on Act production during the stationary phase (especially during the late stationary phase). It can be seen that the TAG pool is the most important intracellular carbon source when the external carbon source is depleted and Act is produced.

The dynamic change trend of TAG pool is common in Streptomyces bacteria: Based on the glucose consumption, Act synthesis and time-course change of TAG pool of M145 in a closed culture system, it can be found that the amount of TAG pool inside the bacteria shows an upward trend during the primary metabolism phase and a downward trend during the Act synthesis phase (FIG. 2c). Based on the time-course metabolomics data of M145, metabolites that are closely related to glucose consumption during the primary metabolism phase (20-48h) and metabolites that are closely related to Act production during the late stationary phase (72-96h) (characterized by the correlation with the change trend of Act concentration) are identified, so as to study the dynamic relationship of TAG and carbon input (glucose consumption) and output (production of polyketide compounds), the compounds closely related to the generation of Act are identified. As shown in FIG. 2d, 11 of the 20 metabolites that are closely and negatively correlated with extracellular glucose contents (i.e., positively correlated with glucose consumption) during the primary metabolism phase (20-48h) are TAG fatty acid moieties (r²>0.8, p value<0.001), indicating that the consumption of glucose during the primary metabolism phase leads to the accumulation of TAG During the late stationary phase (when external glucose is depleted), the 35 metabolites closely related to Act production contain almost all TAG fatty acid moieties (14 of the 15 TAG fatty acid moieties) (r²>0.8, p value<0.001) (FIG. 2d), indicating that the contribution of TAG pools to Act production is much greater than other metabolites. Time-course metabolome analysis of HY01 has also obtained similar results: fatty acid moieties of all TAGs accumulate during the primary metabolism phase, and decreases in the polyketide synthesis phase. In particular, 13 of the 18 compounds that contribute the most to Act the production during the stationary phase are TAG fatty acid moieties. This indicates that TAG pool plays an important role in the switching from primary metabolism to synthesis polyketide compounds.

The expression of genes related to TAG metabolism is analyzed (see Li et al., 2015, SciRep, 5:15840 for analysis method) by profiling the time-course transcriptome of M145 in the same culture condition (Gene Expression Omnibus (GEO) no. GSE53562). As shown in FIGS. 2e-2f, transcription of genes for TAG biosynthesis is upregulated during the primary metabolism phase, and then is downregulated. During the polyketide production phase, genes of β-oxidation pathway related to TAG decomposition is upregulated, which is also consistent with TAG metabolic profile (FIG. 1e). Analysis of transcriptome data (data of GSE2983 (M145) in R5 medium, data of GSE18489 (M145) in fermentation medium, and data of GSE30570 (M145) and GSE30569 (glnK mutant of M145) in SSBM-E medium) (Li, S., et al., Sci. Rep. 5, 15840, 2015) under different culture conditions in the previous studies show that the trends of the transcription profile of these genes are similar. In addition, time-course changes of TAG pool in different industrial strains of Streptomyces avermitilis A56 (Zhuo et al., 2009, PNAS, 107: 11250-11254), Streptomyces bingchenggensis BC0410 (Zhang et al., 2016, Microb Cell Fact, 15: 152) and Streptomyces rimosus (Yin et al., 2015, Microb Cell Fact, 14: 46) are analyzed, the results indicate that TAG pools show similar metabolic trends, that is, TAG is accumulated during the primary metabolism phase and TAG degraded during the secondary metabolism stage (FIG. 2c). All these results indicate that the dynamic trends of TAG pool are common in Streptomyces bacteria.

TAG Degradation Affects Metabolic Flux of Strains

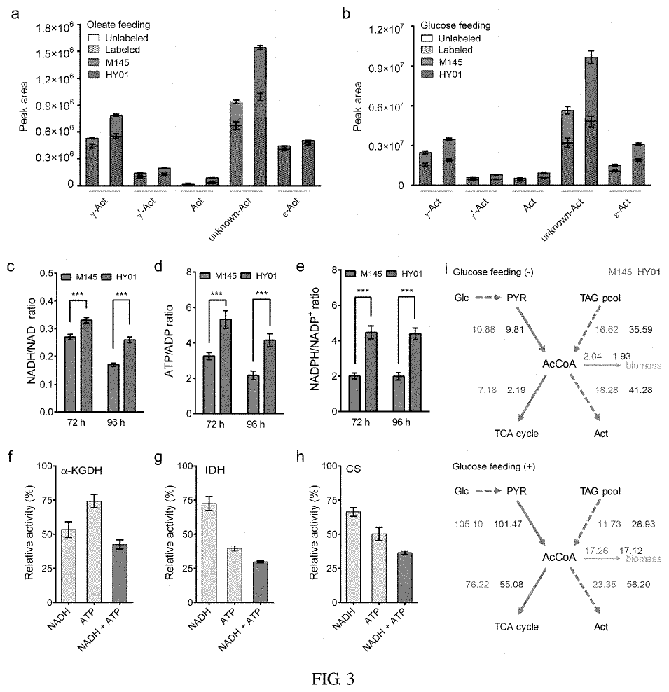

As mentioned above, there is a great difference in Act production between M145 and HY01 during the stationary phase, which is mainly due to the fact that more TAG is degraded during the stationary phase of HY01. In order to further confirm this point, the source of carbon atoms in Act during the stationary phase is traced by using stable isotope (¹³C) labeling. Experiments of fully labeled oleic acid ([U-¹³C]-oleic acid) and fully labeled glucose ([U-¹³C]-glucose) show that both TAG pool and glucose contribute significantly to Act synthesis. In addition, TAG degradation pathway is found more active in high-yielding Act strains (FIGS. 3a-b). In [U-¹³C]-oleic acid labeling experiment and ([U-¹³C]-glucose) labeling experiment, we find that, in addition to α-ketoglutarate, both the concentrations of labeled and unlabeled TCA intermediate metabolites in HY01 are lower than that of the corresponding intermediate metabolites in M145 under two conditions, indicating that the activity of α-ketoglutarate dehydrogenase in the citric acid cycle (TCA) may be inhibited. Previous study (Vemuri et al., PNAS, 2007, 104: 2402-2407) shows that high concentrations of reducing power (NADH) and ATP can inhibit the activities of citrate synthase, isocitrate dehydrogenase and α-ketoglutarate dehydrogenase. Our experimental data show that the intracellular reducing power level (NADH/NAD⁺) and ATP are significantly higher than that in high-yielding strain HY01 in M145 (FIGS. 3c-e). In vitro experiments, the activity of citrate synthase, isocitrate dehydrogenase and α-ketoglutarate dehydrogenase in cell homogenates with or without adding exogenous NADH and/or ATP are shown in FIGS. 3f-h. The activities of the three enzymes are reduced by 63.7%, 71.3% and 57.5% respectively under the synergistic effect of NADH and ATP (FIGS. 3f-h). Based on the above data, it is hypothesized that TAG decomposition (via β-oxidation) produces more reducing equivalents (NADH and FADH), resulting in changing in the distribution of HY01 metabolic carbon flux, so that the carbon flux toward TCA cycle is decreased, and the carbon flux toward Act biosynthetic pathway is increased. In order to verify this hypothesis, carbon metabolic flux analysis (MFA) during the Act stationary phase is conducted by using flux-balance analysis and metabolic network model (Borodina, I., etc., Genome-scale analysis of Streptomyces coelicolor A3 (2) metabolism. Genome Res 15,820-829(2005)) of Streptomyces coelicolor. The results show that the carbon flux of TAG degradation pathway and Act biosynthesis pathway in the high-yielding strain HY01 is significantly higher than that in M145, and the carbon flux from acetyl-coenzyme A to TCA cycle is significantly lower than that in M4 (FIG. 3i). These results indicate that the intracellular TAG pool not only provides a precursor for the synthesis of polyketide compounds during the stationary phase, but also acts as a regulator to regulate the redistribution of carbon metabolic flux during the stationary phase to ensure efficient synthesis of polyketide compounds.

Identification of Key Genes Affecting TAG Pool Degradation

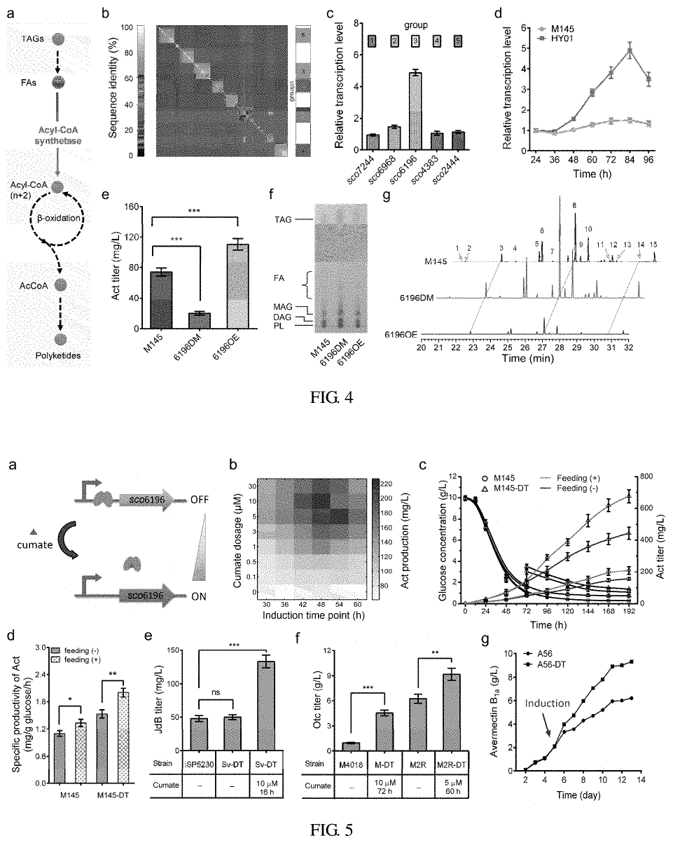

It is observed that about 6-85% of different TAG fatty acid moieties still exist in M145 at the end of the fermentation. As mentioned above, due to the fact that TAG pool provides carbon sources for the generation of secondary metabolites during the stationary phase and regulates the metabolic pathway, it is hypothesized that that regulating TAG decomposition at a specific time during the stationary phase can increase the production of polyketide compounds. To verify this, it is envisaged that acyl-coenzyme A synthetase can be manipulated to carry out the timing regulation of TAG decomposition in M145 (FIG. 4a). It is known that the substrate specificities of acyl-coenzyme A synthetases of Escherichia coli (FadD) and Bacillus subtilis (LcfA and YhfL) are the most extensive (Kameda, K. & Nunn, Journal of Biological Chemistry 256, 5702-5707, 1981); Matsuoka, H. et al., J Biol Chem 282, 5180-5194, 2007). With FadD, LcfA and YhfL as targets, the genomes of 125 fully sequenced Streptomyces strains are searched, among which, homologous proteins of 888 acyl-coenzyme A synthases are compared pairwise and clustered by using Needman-Winsch algorithm (Rice, P., etc., EMBOSS: The European Molecular Biology Open Software Suite. Trends Genet 16,276-277 (2000)), and five different types of acyl-coenzyme A synthases in Streptomyces coelicolor are screened (FIG. 4b), and five different types of acyl-coenzyme A synthases in Streptomyces coelicolor are screened by pairwise comparison and cluster analysis using Nidman-Warm global comparison algorithm (SCO7244 (GI: 2122521), SCO6968 (GI: 21225255), SCO6196 (GI: 21224520), SCO4383 (GI: 8897733) and SCO2444 (GI: 21220908)) (FIG. 4b). The transcription levels of these five genes during the stationary phase are analyzed by fluorescence quantitative PCR, and the results show that sco6196 has the highest transcription abundance of (FIG. 4c). Time-course transcriptions of this gene in M145 and HY01 are also analyzed by fluorescence quantitative PCR, and the results show that the transcription level of this gene in HY01 is significantly higher than its transcription level in M145 during the stationary phase (FIG. 4d). Furthermore, overexpression and knockout are used to verify the effect of this gene on Act biosynthesis. The results show that the Act production in sco6196 deletion mutant (6196DM) is significantly reduced, while the Act production in sco6196 overexpression strain (61960E) is significantly increased (FIG. 4e). Correspondingly, both the results of TLC and GC-MS show that the TAG pool content in 6196DM increases during the stationary phase while the TAG pool content in 61960E decreases when compared with M145 (FIGS. 4f-g). These results all indicate that the gene sco6196 is a key gene affecting intracellular TAG pool the degradation and Act production.

Universality of the strategy of increasing production of polyketide compounds by controlling the timing of TAG decomposition.

Furthermore, a strategy is proposed to increase the production of polyketide compounds by using an induced expression system to control the expression of gene sco6196 and TAG pool degradation in a real-time quantitative manner (FIG. 5a). Sco6196 is cloned into a cumate induction system (Horbal, L., etc., Applied Microbiology and Biotechnology 98, 8641-8655, 2014) to obtain a pCu-SCO6196 plasmid, the pCu-SCO6196 plasmid is transformed into M145 to obtain an engineered strain M145-DT. Under optimized induction conditions (adding 10 μM cumate at 48 h, FIG. 5b), the Act production of M145-DT is 216.1±15.1 mg/L, which is 1.63-fold higher than that of the parental strain M145 and 58% higher than the high-yielding strain HY01 (FIG. 5c), and the specific productivity of Act of the strain is also significantly improved (FIG. 5d).

The TAG decomposition real-time control module constructed as above is integrated into the genome of Streptomyces venezuelae ISP5230 to obtain an engineered strain Sv-DT. ISP5230 strain is used to produce Jedomycin B (JdB). The productions of JdB at different induction concentrations (0.1-30 μM) and different induction times (8-24h) are measured, and the best induction condition is to add 10 μM cumate in the medium at 16 h. Under this condition, Sv-DT produces 133.0±9.4 mg/L of JdB at 48 h, which is 1.7-fold higher than the parent strain ISP5230 (FIG. 5e).

The TAG decomposition real-time control module constructed as above is integrated into the genomes of Streptomyces rimosus M4018 and its high-yielding engineered strain M2R to obtain engineered strains M-DT and M2R-DT, respectively. M4018 strain is used to produce oxytetracycline (Otc). The productions of Otc at different induction concentrations (0.1-30 μM) and different induction times (48-96 h) are measured, and the best induction condition for the two strains (M-DT and M2R-DT) are to add 10 μM cumate in the medium at 72 h and 5 μM cumate at 60 h, respectively. Under this condition, the productions of the two strains are increased by 3.7-fold to reach 4.54±0.58 g/L and by 48% to reach 9.17±0.82 g/L (FIG. 5f).

The above results all indicate that real-time control of TAG decomposition can improve fermentation productions on a laboratory scale. Furthermore, the effect of this strategy is evaluated on an industrial scale (such as a stirred tank bioreactor). The TAG decomposition real-time control module constructed as above is integrated into the genome of Streptomyces avermitilis to obtain an engineered strain A56-DT. The TAG decomposition of A56-DT is real-time controlled in a 180 m³ fermentor, and A56-DT produces 9.31 g/L of abamectin B1a (FIG. 5g), which is the highest output that can be achieved on an industrial scale currently reported. These results all indicate that the carbon flux of secondary metabolism can be adjusted by regulating TAG decomposition, so as to increase the fermentation production of polyketide compounds on an industrial scale.

Method

Construction method of TAG degradation regulation module and engineering bacteria M145-DT, Sv-DT, M-DT, M2R-DT, A56-DT, 61960E and 6196DM. Plasmid pGCymRP21 (Horbal et al., 2014, Appl Microbiol Biotechnol, 98:8641-8655) is used as a template, and the cumate inducible promoter is amplified by using primer pairs CuF and CuR. Genome of Streptomyces coelicolor M145 is used as a template, and gene sco6196 is amplified by using primer pairs 6196F and 6196R. These two fragments are subjected to Gibson assembled with pSET152 linear fragment (Bierman et al., 1992, Gene, 116:43-49) digested with restriction enzymes XbaI/EcoRV by using a Gibson method, and plasmid pCu-SCO6196 is obtained, namely TAG degradation control module. This plasmid can be used to control degradation of the intracellular TAG in a real-time quantitative manner. The plasmid is integrated into genomes of Streptomyces coelicolor M145, Streptomyces venezuelae ISP5230, Streptomyces rimosus M4018 and M2R, and Streptomyces avermitilis industrial strain A56 by conjugation transfer method (Tobias et al., 2000, Practical Streptomyces Genetics, The John Inns Foundation, Norwich, UK) to obtain strains M145-DT, Sv-DT, M-DT, M2R-DT and A56-DT, respectively.

Construction of 61960E: firstly, M145 is used as a template, and gene sco6196 is amplified by using primer pairs 96F1 and 96R1. The amplified product is ligated with plasmid plMEP (Wang et al., 2014, Proc Natl Acad Sci USA 111, 5688-5693) digested with EcoRV and EcoRI by using a Gibson method, and plasmid pIMEP-960E is obtained. This plasmid is integrated into the genome of M145 strain by conjugation transfer method to obtain 61960E strain.

6196DM is constructed by homologous recombination. Firstly, the plasmid backbone of PKC 1132 (Tobias et al., 2000, Practical Streptomyces Genetics, The John Inns Foundation, Norwich, UK) is amplified by using primers 1132F and 1132R, then the upper and lower arms of gene sco6196 are amplified by using 96LF and 96LR, and 96RF and 96RR, respectively. The three amplified fragments are ligated together to obtain plasmid pKC1132-96DM. This plasmid is integrated into the genome of M145 strain by conjugation transfer method, and a homologous double-exchange strain 6196DM is obtained by screening.

The primer sequences used for strain construction are shown in Table 2.

Analytical Method

All analyzed samples are provided for 3 or 5 biological replicates.

Determination of intracellular metabolites of Streptomyces coelicolor by GC-MS.

Cultures collected at different fermentation times are collected and pretreated as follows. (1) Bacteria in the fermentation broth are quickly collected and filtered (acetyl cellulose membrane having a pore size of 0.8 μm, filtration time<30 s), and washed with 30 ml of deionized water precooled to 0° C. (2) The collected cells are quickly transferred to a mortar and immersed in liquid nitrogen (30 s) for quenching. If the following steps cannot be performed immediately, the cells can be frozen at −80° C. for several weeks. (3) The frozen cells are ground into powders in liquid nitrogen. (4) The cell powders (200 mg) are quickly suspended in 1.0 ml of 50v/v % methanol/water extraction buffer pre-cooled to −20° C., and vortexed and mixed in a cold ethanol bath at −20° C. for 30 s. The mixture is subjected to three freeze-thaw cycles (45 s/3 min for each cycle). After centrifugation at the condition of −15° C. and 13,000×g for 10 min, the supernatant (0.8 ml) is collected, and the precipitate is re-extracted twice by adding 0.5 ml of extraction buffer, then the extracts are combined. 100 μL of succinic-d₄ acid (0.14 mg/ml) used as an internal standard is added to a total of 1.3 ml of metabolite extract for correction. (5) The extract is freezed and freeze-dried overnight in a vacuum concentrator. (6) Methoxyamine hydrochloride is dissolved in pyridine at a concentration of 20 mg/ml before use (Winder, C. L., etc., Anal. Chem. 80, 2939-2948, 2008). For each sample, 60 μL of methoxyamine hydrochloride solution is added and incubated in a water bath at 40° C. for 90 min. The samples are vortexed and mixed for 30 s every 15 min of incubation. (7) N-methyl-N-(trimethylsilyl)-trifluoroacetamide (MSTFA) (80 μL) is added and the sample is incubated under the same conditions as (6). (8) The sample is centrifuged at 13,000×g for 10 min, and GC-MS analysis is performed within 24 h.

Glucose: 1 ml of fermentation broth is centrifuged at 12000 rpm for 10 min, then supernatant of the sample is diluted, and glucose in the fermentation broth is analyzed by using an enzyme analyzer (type SBA-40C).

Malonyl-CoA and acetyl-coenzyme A: The above quenched and ground bacterial powders are extracted by a trichloroacetic acid-ether extraction method. The specific method is as follows: 1.3 ml of trichloroacetic acid pre-cooled by ice-water is added to 200 mg of bacterial grinding materials, which was shaken at 0° C. for 3 min, followed by centrifuging at 0° C. for 10 min, then the supernatant is collected, and 2 ml of pre-cooled (−20° C.) ether is added to remove trichloroacetic acid. The aqueous phase is recovered and freeze-dried, and the freeze-dried product is re-dissolved in 300 μL of ice-precooled ammonium formate (25 mM). The malonyl-CoA and acetyl-coenzyme A are detected by LC-MS/MS after passing through a membrane (0.22 μm).

Act: 1 mL of fermentation broth is collected, and the bacteria are collected by centrifuging at 4° C. and 10000×g, then 1 mol/L KOH is added to continue to centrifuge to obtain a supernatant, and OD640 is measured by using a microplate reader. Act production is calculated according to the molar absorption coefficient (ε640=25320) at this wavelength.

Gedomycin B, oxytetracycline and abamectin B1a: Gedomycin B (Chen et al., 2005, J Biol Chem, 280: 22508-22514), oxytetracycline (Yin et al., 2015, Microb Cell Fact, 14: 46) and abamectin B1a (Zhuo et al., 2010, PNAS, 107: 11250-11254) are detected according to the method reported previous literature.

Measurement method of biomass: 1 mL of fermentation broth is collected and centrifuged, then the supernatant is removed, and the precipitate is washed with SET buffer once, then centrifuging is performed, and the supernatant is removed. The bacteria are re-suspended in 2 mL of diphenylamine reagent, and bathed in water at 60° C. for 1 h, followed by centrifuging, and the supernatant is determined for OD595.

TAG analysis: The fermentation broth is collected, and the bacteria are collected by centrifuging at −9° C. and 13000×g for 1 min. Then the cells are snap frozen in liquid nitrogen for 2 min, and then freeze-dried in a freeze drier. 10 mg of freeze-dried bacteria are extracted with chloroform/methanol (2:1, v/v) in a water bath at 40° C. for 3 h, and shaken vigorously for 1 min every half h. TAG is separated by a thin-layer chromatography chromatographic plate, wherein n-hexane: ether: acetic acid (80:20:1) is used as a developing solvent, and copper phosphate is used as a color developer to develop at 100° C. Tanon 1600 gel imaging system is used for quantitative analysis based on TAG grayscale.

Determination of reducing power: NADH/NAD⁺, ATP/ADP and NADPH/NADP⁺ are determined by using a kit (BioVision, USA) according to the manufacturer's instructions.

Activity determination of citrate synthase, isocitrate dehydrogenase and α-ketoglutarate dehydrogenase in vitro: After 36 h of cultivation in SMM medium, the collected fermentation broth is centrifuged for collecting the bacteria, which are washed with deionized water for 3 times, followed by crushing the cells by ultrasound. The crushed cells are centrifuged, and the supernatant is collected to obtain a crude enzyme solution, which is used to analyze the activities of three different enzymes. To determine the activity of citric acid synthase, add oxaloacetate (0.5 mM), acetyl-coenzyme A (0.2 mM), Tris-HCl (100 mM, pH 7.5), 5,5′-disulfide bis (2-nitrobenzoic acid) and the crude enzyme solution (200 μL) are added to a 2 mL reaction system, and the activity of citrate synthase is determined by measuring the change in the content of citric acid by HPLC. To determine the activity of isocitrate dehydrogenase, Tris-HCl (100 mM, pH 7.5), isocitrate (0.2 mM), manganese sulfate (1.5 mM) and the crude enzyme solution (200 μL) are added to a 2 mL reaction system, and the content of α-ketoglutarate is determined by HPLC, so as to determine the activity of isocitrate dehydrogenase. To determine the activity of α-ketoglutarate dehydrogenase, Tris-HCl (150 mM, pH 8.0), coenzyme A (0.1 mM), thiamine pyrophosphate (0.2 mM), and L-cysteine (2.5 mM), α-ketoglutarate (2.5 mM) and the crude enzyme solution (200 μL) are added to a 2 mL reaction system, and the content change of succinyl-CoA is determined by HPLC-MS/MS, so as to determine the activity of α-ketoglutarate dehydrogenase. To determine the effects of NADH or/and ATP on the activities of three different enzymes, 0.1 mM NADH or/and ATP is added to each reaction system as needed. All reactions are carried out at 30° C. for 15 min.

Isotope labeling analysis: Labelled intracellular metabolites are analyzed by using a multi-reaction monitoring model in LC-MSMS (Han, J. et al. Metabolomic analysis of key central carbon metabolism carboxylic acids as their 3-nitrophenylhydrazones by UPLC/ESI-MS. Electrophoresis 34,2891-2900 (2013)), and natural isotope abundance is corrected (Yuan, J. et al. Kinetic flux profiling for quantitation of cellular metabolic fluxes. Nature Protocols 3, 1328-1340 (2008)).

Fluorescence quantitative PCR analysis: The primers used are shown in Table 3.

TABLE 1
Metabolites identified by GC-MS

| | | Chemical | |
|---|---|---|---|
| Type | Putative metabolite ^{a} | formula | Metabolic pathway |
| Amino acid | L-alanine | C₃H₇NO₂ | Metabolism of |
| metabolism | L-aspartic acid | C₄H₇NO₄ | alanine, aspartic |
| | N-carbamoyl-L-aspartic acid | C₅H₈N₂O₅ | acid and glutamic |
| | | | acid |
| | 5-aminovaleric acid | C₅H₁₁NO₂ | Metabolism of |
| | DL-ornithine | C₅H₁₂N₂O₂ | arginine and proline |
| | L-citrulline | C₇H₁₄N₂O₃ | |
| | L-proline | C₅H₉NO₂ | |
| | L-glutamic acid 5-phosphate | C₅H₁₀NO₇P | |
| | N-acetyl-L-glutamic acid | C₇H₁₁NO₅ | |
| | Trans-4-hydroxy-L-proline | C₅H₉NO₃ | |
| | N-acetyl-L-glutamic acid | C₇H₁₂NO₈P | |
| | 5-phosphate | | |
| | L-glutamine | C₅H₁₀N₂O₂ | Metabolism of |
| | | | D-glutamine and |
| | | | D-glutamic acid |
| | 5-oxo-L-proline | C₅H₇NO₃ | Glutathione |
| | L-cysteine | C₃H₇NO₂S | metabolism |
| | Glycine | C₂H₅NO₂ | Metabolism of |
| | L-threonine | C₄H₉NO₃ | glycine, serine and |
| | 2-oxobutyric acid | C₄H₆O₃ | threonine |
| | Serine | C₃H₇NO₃ | |
| | 3-phosphoserine | C₃H₈NO₆P | |
| | L-2-amino-3-oxobutyric acid | C₄H₇NO₃ | |
| | 2-oxohexanedioic acid | C₆H₈O₅ | Leucine and |
| | Glutaric acid | C₅H₈O₄ | tryptophan |
| | Pipecolic acid | C₆H₁₁NO₂ | |
| | Phenylalanine | C₉H₁₁NO₂ | Phenylalanine |
| | Phenylpyruic acid | C₉H₈O₃ | metabolism |
| | 3,4-dihydroxyphenylpyruvic acid | C₉H₈O₅ | Tyrosine |
| | Tyrosine | C₉H₁₁NO₃ | metabolism |
| | 3-hydroxy-L-tyrosine | C₉H₁₁NO₄ | |
| | 3-hydroxy-3-methyl-2-oxobutyric | C₅H₈O₄ | Biosynthesis of |
| | acid | | valine, leucine and |
| | | | isoleucine |
| | 2-Oxoisovaleric acid | C₅H₈O₃ | Metabolism of |
| | L-isoleucine | C₆H₁₃NO₂ | valine, leucine and |
| | L-leucine | C₆H₁₃NO₂ | isoleucine |
| | 4-methyl-2-oxovalericacidethylester | C₆H₁₀O₃ | |
| | L-valine | C₅H₁₁NO₂ | |
| Metabolism of | Hydroxymethylphosphonic acid | CH₅O₄P | Metabolism of |
| other amino | | | phosphonic acid |
| acids | | | and |
| | | | hypophosphorous |
| | | | acid |
| Carbohydrate | D-glyceric acid-3-phosphoric acid | C₃H₇O₇P | Glycolysis/ |
| metabolism | D-galactose | C₆H₁₂O₆ | gluconeogenesis |
| | 1,3-diphospho-D-glyceric acid | C₃H₈O₁₀P₂ | |
| | Dihydroxyacetone phosphate | C₃H₇O₆P | |
| | 3-phosphoglyceraldehyde | C₃H₇O₆P | |
| | Glucose-6-phosphatase | C₆H₁₃O₉P | |
| | Lactic acid | C₃H₆O₃ | |
| | Pyruvic acid | C₃H₄O₃ | |
| | Citric Acid | C₆H₈O₇ | Tricarboxylic acid |
| | DL-malic acid | C₄H₆O₅ | cycle |
| | Oxaloacetic acid | C₄H₄O₅ | |
| | Fumaric acid | C₄H₄O₄ | |
| | Isocitric acid | C₆H₈O₇ | |
| | Succinic acid | C₄H₆O₄ | |
| | α-Ketoglutaric acid | C₅H₆O₅ | |
| | 6-phospho-D-gluconic acid | C₆H₁₃O₁₀P | Pentose phosphate |
| | 6-phosphogluconic acid | C₆H₁₃O₁₀P | pathway |
| | D-pentahydroxy-heptulose | C₇H₁₅O₁₀P | |
| | 7-phosphate | | |
| | D-ribose-5-phosphate | C₅H₁₁O₈P | |
| | D-ribose | C₅H₁₀O₅ | |
| | D-erythrose-4-phosphate | C₄H₉O₇P | |
| | D-ribulose 5-phosphate | C₅H₁₁O₈P | |
| | D-xylulose 5-phosphate | C₅H₁₁O₈P | |
| | Ribitol | C₅H₁₂O₅ | Mutual conversion |
| | DL-arabinose | C₅H₁₀O₅ | of pentose and |
| | D-ribulose | C₅H₁₀O₅ | glucuronic acid |
| | D-xylofuranose | C₅H₁₀O₅ | |
| | D-xylopyranose | C₅H₁₀O₅ | |
| | Galacturonic acid | C₆H₁₀O₇ | |
| | Glycidaldehyde | C₃H₆O₃ | |
| | Lyxose | C₅H₁₀O₅ | |
| | Xylitol | C₅H₁₂O₅ | |
| | L-fucose-1,5-lactone | C₆H₁₀O₅ | Galactose |
| | D-fructose | C₆H₁₂O₆ | metabolism |
| | D-fructose-1,6-diphosphate | C₆H₁₄O₁₂P₂ | |
| | D-mannitol | C₆H₁₄O₆ | |
| | D-mannopyranose-6-phosphate | C₆H₁₃O₉P | |
| | D-mannose 6-phosphate | C₆H₁₃O₆P | |
| | L-fucose acid | C₆H₁₂O₆P | |
| | D-mannose | C₆H₁₂O₆ | |
| | L-furarhamnose | C₆H₁₂O₅ | |
| | L-rhamnose 1-phosphate | C₆H₁₃O₈P | |
| | Mannitol phosphate | C₆H₁₅O₉P | |
| | Sorbitol | C₆H₁₄O₆ | |
| | D-galactose | C₆H₁₂O₆ | Metabolism of |
| | D-galactopyranose | C₆H₁₂O₆ | galactose |
| | D-galactopyranosyl 1-phosphate | C₆H₁₃O₉P | |
| | D-fructofuranose 6-phosphate | C₆H₁₃O₉P | |
| | Lactose | C₁₂H₂₂O₁₁ | |
| | D-galactonic acid | C₆H₁₂O₇ | |
| | Melibiose | C₁₂H₂₂O₁₁ | |
| | Sucrose | C₁₂H₂₂O₁₁ | |
| | D-glucuronolactone | C₆H₈O₆ | Ascorbate and |
| | L-arabinose-1,4-lactone | C₅H₈O₅ | aldarate metabolism |
| | 4-hydroxybutanoic acid | C₄H₈O₃ | Butanoate |
| | | | metabolism |
| | 2-oxovaleric acid | C₅H₈O₃ | Pyruvate |
| | | | metabolism |
| | Glyoxylic acid | C₂H₂O₃ | Glyoxylate and |
| | Glycolic acid | C₂H₄O₃ | dicarboxylate |
| | Oxalic acid | C₂H₂O₄ | metabolism |
| | Myo-inositol | C₆H₁₂O₆ | Phosphoinositide |
| | Inositol monophosphate | C₆H₁₃O₉P | metabolism |
| | Inositol | C₆H₁₂O₇ | |
| | Scyllitol | C₆H₁₂O₇ | |
| | Maltose | C₁₂H₂₂O₁₁ | Starch and sucrose |
| | | | metabolism |
| Digestive | 2-methylpropionic acid | C₄H₈O₂ | Protein digestion |
| system | | | and absorption |
| Energy | Phosphoric acid | H₃O₄P | Oxidative |
| metabolism | | | phosphorylation |
| Lipid | Dodecanoic acid | C₁₂H₃₀O₂ | Fatty acid |
| metabolism | Myristic acid | C₁₄H₂₈O₂ | biosynthesis |
| | Palmitic acid | C₁₆H₃₂O₂ | |
| | Heptadecanoic acid | C₁₇H₃₄O₂ | |
| | Stearic acid | C₁₈H₃₆O₂ | |
| | n-Pentadecanoic acid | C₁₅H₃₀O₂ | |
| | MG (16:0/0:0/0:0) | C₁₉H₃₈O₂ | Glycerolipid |
| | MG (18:0/0:0/0:0) | C₂₁H₄₂O₄ | metabolism |
| | Glyceraldehyde-3-phosphate | C₃H₉O₆P | |
| | Glycerol | C₃H₈O₃ | |
| Transmembrane | Methyl-P-D-galactopyranoside | C₇H₁₄O₆ | ATP-binding |
| transport | | | cassette transporter |
| Metabolism of | Nicotinic acid | C₆H₅NO₂ | Nicotinic acid and |
| cofactors and | Quinolinic acid | C₇H₅NO₄ | nicotinamide |
| vitamins | | | metabolism |
| | Retinoic acid | C₂₀H₂₈O₂ | Retinol metabolism |
| Nucleotide | Adenine | C₅H₅N₅ | Purine metabolism |
| metabolism | Aminoformic acid | CH₃NO₂ | Pyrimidine |
| | Carbamyl phosphate | CH₄NO₅P | metabolism |
| | Uracil | C₄H₄N₂O₂ | |
| | Thymine | C₅H₆N₂O₂ | |
| | Thymidine | C₁₀H₁₄N₂O₅ | |
| | Propanedioic acid | C₃H₄O₄ | |
| | Xanthine | C₅H₄N₄O₂ | |
| | 5-phosphoribosyl amine | C₅H₁₂NO₇P | |
| | Xanthosine | C₁₀H₁₂N₄O₆ | |
| | Guanine | C₅H₅N₅O | |
| | Uridine | C₉H₁₂N₂O₆ | |
| | Uridine 5′-monophosphate | C₉H₁₃N₂O₉P | |
| Other | Adipic acid | C₆H₁₀O₄ | |
| metabolic | 2-Phenylbutyric acid | C₁₀H₁₂O₂ | |
| pathways | 3-Hydroxypyridine | C₅H₅NO | |
| | 3-Hydroxy-tetradecanedioic acid | C₁₄H₂₆O₅ | |
| | Deoxyribonucleic acid lactone | C₅H₈O₄ | |
| | D-erythritol | C₄H₁₀O₄ | |
| | D-erythrose | C₄H₈O₄ | |
| | D-turanose | C₁₂H₂₂O₁₁ | |
| | Estradiol methyl ether | C₁₉H₂₆O₂ | |
| | Heneicosane | C₂₁H₄₄ | |
| | L-norvaline | C₅H₁₁NO₂ | |
| | N-acetylglucosamine | C₈H₁₅NO₆ | |
| | Valeric acid | C₅H₁₀O₂ | |
| | N-α-Acetyl-L-lysine | C₈H₁₆N₂O₃ | |
| | Succinylacetone | C₇H₁₀O₄ | |

^{a} The putative metabolites are identified by searching the commercial database National Institute of Standards and Technology (NIST 8.0) Mass Spectrometry Library and Fiehn Metabolism Library³. Metabolites with indications greater than 80% are identified directly. For metabolites with indications between 40% and 80%, manual correction is performed by comparing the measured mass spectrum with the putative compound in NIST8.0. Metabolites indicated below 40% are ignored.

TABLE 2
Primers used in plasmid construction

| Primers | | |
|---|---|---|
| Description | Name | Sequence (5′-3′) |
| Construction | CuF | Aagcttgggctgcaggtcgactctagagttat |
| of | | caccgcttgaacttggc (SEQ ID No: 1) |
| pCu- | CuR | gacggctgggggcgcggggtgcggtcactggg |
| SCO6196 | | gtcctcctgttgctcgactagtataatac |
| | | (SEQ ID NO: 2) |
| | 6196F | gtgaccgcacccgcgccagccgtc |
| | | (SEQ ID NO: 3) |
| | 6196R | Acatgattacgaattcgatatcgcgcggccgc |
| | | ggatctcaggggcgcgctccgtacc |
| | | (SEQ ID NO: 4) |
| Confirmation | LCF | gccaagttcaagcggtgataatctagacgctc |
| of | | cctgcccgctatggtgacga |
| pCu- | | (SEQ ID NO: 5) |
| SCO6196 | LCR | ccctgatgataagcattacg |
| | | atatcgaattcgtaatcatgtcatagctg |
| | | (SEQ ID NO: 6) |
| Construction | 96LF | cagtgccaagcttgggctgcaggagaacggtc |
| of 6196DM | | cggcgattgtcctcg |
| | | (SEQ ID NO: 7) |
| | 96LR | cgagtgggtcctcgtccagtacgggatccagg |
| | | agttcctgtacgcccacc |
| | | (SEQ ID NO: 8) |
| | 96RF | tcctggatcccgtactggacgaggacccactc |
| | | g (SEQ ID NO: 9) |
| | 96RR | ctatgacatgattacgaattcgatatcgatga |
| | | aactgcgcgcggtc |
| | | (SEQ ID NO: 10) |
| | 1132F | catcgatatcgaattcgtaatcatgtcatag |
| | | (SEQ ID NO: 11) |
| | 1132R | gttctcctgcagcccaagcttggcactggc |
| | | (SEQ ID NO: 12) |

| | | |
|---|---|---|
| Confirmation | 96vF | agtaggcgcgcagttcctccag |
| of 6196DM | | (SEQ ID NO: 13) |
| | 96vR | tcccgtccggacggcgctggacctacg |
| | | (SEQ ID NO: 14) |
| Construction | 96F1 | atctagcggaacggatctag ag atgtg |
| of 61960E | | accgcacccgcgccccagccgtc |
| | | (SEQ ID NO: 15) |
| | 96R1 | ttccatcgccgcttcatgatgaattctcgccgc |
| | | ggccgataccggtgc |
| | | (SEQ ID NO: 16) |

TABLE 3
Primers used in fluorescent quantitative PCR

| Description | Name | Sequence (5'-3') |
|---|---|---|
| sco6196 | 96F2 | ggcatctgggcggtcaact |
| | | (SEQ ID NO: 17) |
| | 96R2 | gctgctcttgtggggcgagg |
| | | (SEQ ID NO: 18) |
| Comparison | 0710F | tgtccgccctccgctccgtgtgtcc |
| | | (SEQ ID NO: 19) |
| | 0710R | tccaggaccgtgtcgccgtag |
| | | (SEQ ID NO: 20) |
| sco7244 | 7244F | gtgcagctcctgtacacctc |
| | | (SEQ ID NO: 21) |
| | 7244R | ctcaggtactcgtgcaccag |
| | | (SEQ ID NO: 22) |
| sco6968 | 6968F | cagaccgtctccctcaactc |
| | | (SEQ ID NO: 23) |
| | 6968R | cctccttcaccatcagctcg |
| | | (SEQ ID NO: 24) |
| sco4383 | 4383F | catccagaaccaccgcatca |
| | | (SEQ ID NO: 25) |
| | 4383R | tcagcgaggagaggtcgtag |
| | | (SEQ ID NO: 26) |
| sco2444 | 2444F | agagcggcggttacaagatc |
| | | (SEQ ID NO: 27) |
| | 2444R | cacgatccgttccccgag |
| | | (SEQ ID NO: 28) |

1. A method for improving the production of a polyketide compound in a Streptomyces, comprising a step of strengthening a triacylglycerol decomposition pathway in a Streptomyces during a stationary phase.

2. The method of claim 1, wherein the triacylglycerol decomposition pathway is a β-oxidation pathway.

3. The method of claim 1, wherein the polyketide compound is selected from the group consisting of a type I polyketone compound, a type II polyketone compound, and a type III polyketone compound.

4. The method of claim 3, wherein the polyketide is selected from the group consisting of actinomycin, jadomycin, avermectin, milbemycin, oxytetracycline and nemadectin.

5. The method of claim 1, wherein a fatty acid moiety of the triacylglycerol is a fatty acid having a carbon number of 12-24.

6. The method of claim 1, wherein the Streptomyces is selected from the group consisting of a Streptomyces coelicolor, a Streptomyces albus, a Streptomyces venezuelae, Streptomyces lividans, a Streptomyces avermitilis, a Streptomyces rimosus, a Streptomyces hygroscopicus, a Streptomyces cyaneogriseus, and a Streptomyces bingchenggensis.

7. The method of claim 2, wherein the triacylglycerol decomposition pathway is strengthened by enhancing an expression level and/or activity of at least one enzyme in the Streptomyces that catalyzes an irreversible reaction of the β-oxidation pathway.

8. The method of claim 7, wherein the enzyme that catalyzes an irreversible reaction of the β-oxidation pathway is selected from the group consisting of an acyl coenzyme A synthetase, an acyl-coenzyme A dehydrogenase, an acyl-coenzyme A hydratase, and any combination thereof.

9. The method of claim 8, wherein the Streptomyces is a Streptomyces coelicolor, the acyl coenzyme A synthetase is selected from the group consisting of SCO1330, SCO2131, SCO2444, SCO2561, SCO2720, SCO3436, SCO4006, SCO4503, SCO5983, SCO6196, SCO6552, SCO6790, SCO6968, SCO7244, SCO7329, SCO4383, and any combination thereof; the acyl-coenzyme A dehydrogenase is selected from the group consisting of SCO1690, SCO2774, SCO6787, and any combination thereof; and the acyl-coenzyme A hydratase is selected from SCO4384 and/or SCO6732.

10. The method of claim 8, wherein the Streptomyces is a Streptomyces albus, the acyl coenzyme A synthetase is selected from the group consisting of SLNWT_0050, SLNWT_0304, SLNWT_0327, SLNWT_0598, SLNWT_0621, SLNWT_3453, SLNWT_4291, SLNWT_6199, SLNWT_6951, and any combination thereof; the acyl-coenzyme A dehydrogenase is SLNWT_4686; and the acyl-coenzyme A hydratase is selected from the group consisting of SLNWT_0723, SLNWT_0850, SLNWT_4292, SLNWT_6769, SLNWT_6771, and any combination thereof.

11. The method of claim 8, wherein the Streptomyces is a Streptomyces venezuelae, the acyl coenzyme A synthetase is selected from the group consisting of SVEN_0294, SVEN_0876, SVEN_2231, SVEN_3097, SVEN_4199, SVEN_6078, SVEN_6188, SVEN_6773, SVEN_6774, SVEN_7224, and any combination thereof; the acyl-coenzyme A dehydrogenase is selected from SVEN_0520 and/or SVEN_1293; and the acyl-coenzyme A hydratase is selected from the group consisting of SVEN_0030, SVEN_0204, SVEN_0279, SVEN_1657, SVEN_4200, SVEN_5574, SVEN_5576, SVEN_6413, and any combination thereof.

12. The method of claim 8, wherein the Streptomyces is a Streptomyces lividans, the acyl coenzyme A synthetase is selected from the group consisting of SLIV_03075, SLIV_04410, SLIV_07155, SLIV_16515, SLIV_25480, SLIV_36365, and any combination thereof; the acyl-coenzyme A dehydrogenase is SLIV_29290; and the acyl-coenzyme A hydratase is selected from SLIV_16510 and/or SLIV_36115.

13. The method of claim 8, wherein the Streptomyces is a Streptomyces avermitilis, the acyl coenzyme A synthetase is selected from the group consisting of SAVERM_1258, SAVERM_1346, SAVERM_1603, SAVERM_2030, SAVERM_2279, SAVERM_377, SAVERM_3806, SAVERM_3864, SAVERM_5723, SAVERM_605, SAVERM_6612, and any combination thereof; the acyl-coenzyme A dehydrogenase is selected from the group consisting of SAVERM_1381, SAVERM_5280, SAVERM_6614, and any combination thereof; and the acyl-coenzyme A hydratase is selected from the group consisting of SAVERM_1245, SAVERM_1680, SAVERM_3863, SAVERM_6203, SAVERM_717, SAVERM_7216, and any combination thereof.

14. The method of claim 8, wherein the Streptomyces is a Streptomyces rimosus, the acyl coenzyme A synthetase is selected from the group consisting of an acyl coenzyme A synthetase having a NCBI registration number of WP_053803359.1, ELQ77730.1, WP_033034442.1, KOT44666.1, WP_033033106.1, and any combination thereof; the acyl-coenzyme A dehydrogenase is selected from the group consisting of an acyl coenzyme A dehydrogenase having a NCBI registration number of WP_125057199.1, WP_033031914.1, WP_030661846.1, WP_030634872.1, WP_030370993.1, and any combination thereof; and the acyl-coenzyme A hydratase is selected from an acyl-coenzyme A hydratase having a NCBI registration number of WP_030669923.1 and/or WP_125053679.1.

15. The method of claim 8, wherein the Streptomyces is a Streptomyces bingchenggensis, the acyl coenzyme A synthetase is selected from the group consisting of SBI_00524, SBI_02958, SBI_03178, SBI_04546, SBI_04871, SBI_06310, SBI_07635, SBI_08381, SBI_08662, SBI_09123, and any combination thereof; the acyl-coenzyme A dehydrogenase is selected from SBI_08383 and/or SBI_09842; and the acyl-coenzyme A hydratase is selected from the group consisting of SBI_01088, SBI_01673, SBI_01731, SBI_02642, SBI_04870, and any combination thereof.

16. The method of claim 6, wherein the triacylglycerol decomposition pathway is strengthened by enhancing an expression level and/or activity of an esterase in the Streptomyces.

17. The method of claim 16, wherein the esterase is selected from the group consisting of the following proteins: a Streptomyces coelicolor esterase selected from SCO0713 (NP_625018.1), SCO1265 (NP_625552.1), SCO1735 (NP_626008.1), SCO3219 (NP_627433.1), SCO4368 (NP_628538.1), SCO4746 (NP_628904.1), SCO4799 (NP_628956.1), SCO6966 (NP_631032.1) and SCO7131 (NP_631192.1); a Streptomyces bingchenggensis esterase selected from SBI_00115 (WP_014172715.1), SBI_00631 (WP_014173231.1), SBI_01149 (WP_014173749.1) and SBI_01728 (WP_014174328.1); a Streptomyces avermitilis esterase selected from SAVERM_RS02860 (WP_010981907.1), SAVERM_RS04345 (WP 010036168.1), SAVERM_RS04550 (WP_107083239.1), and SAVERM_RS23405(WP 010985956.1); a Streptomyces albus esterase selected from SLNWT_RS18180 (WP_078845043.1), SLNWT_RS12910 (WP_040249758.1) and SLNWT_RS12900 (WP_040249752.1); and a Bacillus subtilis esterase selected from BSU_08350 (NP_388716.1), BSU_24510 (NP_390331.1) and BSU_21740 (NP_390057.1).

18. The method of claim 8, wherein the enzyme is operably linked to downstream of an inducible promoter and induced during the stationary phase.

19. The method of claim 1, wherein the triacylglycerol decomposition pathway in the Streptomyces is strengthened during the stationary phase by inhibiting a carbon metabolism flow from an acetyl-coenzyme A to a tricarboxylic acid cycle.

20. The method of claim 1, further comprising increasing a NADH/NAD⁺ ratio in the Streptomyces.

21. The method of claim 20, wherein the NADH/NAD⁺ ratio is increased by adding NADH and/or ATP to a medium.

22. A method for switching a primary metabolism to a secondary metabolism in a Streptomyces, comprising strengthening a triacylglycerol decomposition pathway in the Streptomyces.

23. The method of claim 22, wherein the triacylglycerol decomposition pathway is a β-oxidation pathway.

24. The method of claim 22 or 23, wherein a fatty acid moiety of the triacylglycerol is a fatty acid having a carbon number of 12-24.

25. The method of claim 22, wherein the Streptomyces is selected from the group consisting of a Streptomyces coelicolor, a Streptomyces albus, a Streptomyces venezuelae, a Streptomyces lividans, a Streptomyces avermitilis, a Streptomyces rimosus, Streptomyces hygroscopicus, a Streptomyces cyaneogriseus, and a Streptomyces bingchenggensis.

26. The method of claim 22, wherein the triacylglycerol decomposition pathway is strengthened by enhancing an expression level and/or activity of at least one enzyme in the Streptomyces that catalyzes an irreversible reaction of the β-oxidation pathway.

27. The method of claim 26, wherein the enzyme that catalyzes an irreversible reaction of the β-oxidation pathway is selected from the group consisting of an acyl coenzyme A synthetase, an acyl-coenzyme A dehydrogenase, an acyl-coenzyme A hydratase, and any combination thereof.

28. The method of claim 27, wherein the Streptomyces is a Streptomyces coelicolor, the acyl coenzyme A synthetase is selected from the group consisting of SCO1330, SCO2131, SCO2444, SCO2561, SCO2720, SCO3436, SCO4006, SCO4503, SCO5983, SCO6196, SCO6552, SCO6790, SCO6968, SCO7244, SCO7329, SCO4383, and any combination thereof; the acyl-coenzyme A dehydrogenase is selected from the group consisting of SCO1690, SCO2774, SCO6787, and any combination thereof; and the acyl-coenzyme A hydratase is selected from SCO4384 and/or SCO6732.

29. The method of claim 27, wherein the Streptomyces is a Streptomyces albus, the acyl coenzyme A synthetase is selected from the group consisting of SLNWT_0050, SLNWT_0304, SLNWT_0327, SLNWT_0598, SLNWT_0621, SLNWT_3453, SLNWT_4291, SLNWT_6199, SLNWT_6951, and any combination thereof; the acyl-coenzyme A dehydrogenase is SLNWT_4686; and the acyl-coenzyme A hydratase is selected from the group consisting of SLNWT_0723, SLNWT_0850, SLNWT_4292, SLNWT_6769, SLNWT_6771, and any combination thereof.

30. The method of claim 27, wherein the Streptomyces is a Streptomyces venezuelae, the acyl coenzyme A synthetase is selected from the group consisting of SVEN_0294, SVEN_0876, SVEN_2231, SVEN_3097, SVEN_4199, SVEN_6078, SVEN_6188, SVEN_6773, SVEN_6774, SVEN_7224, and any combination thereof; the acyl-coenzyme A dehydrogenase is selected from SVEN_0520 and/or SVEN_1293; and the acyl-coenzyme A hydratase is selected from the group consisting of SVEN_0030, SVEN_0204, SVEN_0279, SVEN_1657, SVEN_4200, SVEN_5574, SVEN_5576, SVEN_6413, and any combination thereof.

31. The method of claim 27, wherein the Streptomyces is a Streptomyces lividans, the acyl coenzyme A synthetase is selected from the group consisting of SLIV_03075, SLIV_04410, SLIV_07155, SLIV_16515, SLIV_25480, SLIV_36365, and any combination thereof; the acyl-coenzyme A dehydrogenase is SLIV_29290; and the acyl-coenzyme A hydratase is selected from SLIV_16510 and/or SLIV_36115.

32. The method of claim 27, wherein the Streptomyces is a Streptomyces avermitilis, the acyl coenzyme A synthetase is selected from the group consisting of SAVERM_1258, SAVERM_1346, SAVERM_1603, SAVERM_2030, SAVERM_2279, SAVERM_377, SAVERM_3806, SAVERM_3864, SAVERM_5723, SAVERM_605, SAVERM_6612, and any combination thereof; the acyl-coenzyme A dehydrogenase is selected from the group consisting of SAVERM_1381, SAVERM_5280, SAVERM_6614, and any combination thereof; and the acyl-coenzyme A hydratase is selected from the group consisting of SAVERM_1245, SAVERM_1680, SAVERM_3863, SAVERM_6203, SAVERM_717, SAVERM_7216, and any combination thereof.

33. The method of claim 27, wherein the Streptomyces is Streptomyces rimosus, the acyl coenzyme A synthetase is selected from the group consisting of an acyl coenzyme A synthetase having a NCBI registration number of WP_053803359.1, ELQ77730.1, WP_033034442.1, KOT44666.1, WP_033033106.1, and any combination thereof; the acyl-coenzyme A dehydrogenase is selected from the group consisting of an acyl coenzyme A dehydrogenase having a NCBI registration number of WP_125057199.1, WP_033031914.1, WP_030661846.1, WP_030634872.1, WP_030370993.1, and any combination thereof; and the acyl-coenzyme A hydratase is selected from an acyl-coenzyme A hydratase having a NCBI registration number of WP_030669923.1 and/or WP_125053679.1.

34. The method of claim 27, wherein the Streptomyces is a Streptomyces bingchenggensis, the acyl coenzyme A synthetase is selected from the group consisting of SBI_00524, SBI_02958, SBI_03178, SBI_04546, SBI_04871, SBI_06310, SBI_07635, SBI_08381, SBI_08662, SBI_09123, and any combination thereof; the acyl-coenzyme A dehydrogenase is selected from SBI_08383 and/or SBI_09842; and the acyl-coenzyme A hydratase is selected from the group consisting of SBI_01088, SBI_01673, SBI_01731, SBI_02642, SBI_04870, and any combination thereof.

35. The method of claim 22, wherein the triacylglycerol decomposition pathway is strengthened by enhancing an expression level and/or activity of an esterase in the Streptomyces.

36. The method of claim 35, wherein the esterase is selected from the group consisting of the following proteins: a Streptomyces coelicolor esterase selected from SCO0713 (NP_625018.1), SCO1265 (NP_625552.1), SCO1735 (NP_626008.1), SCO3219 (NP_627433.1), SCO4368 (NP_628538.1), SCO4746 (NP_628904.1), SCO4799 (NP_628956.1), SCO6966 (NP_631032.1) and SCO7131 (NP_631192.1); a Streptomyces bingchenggensis esterase selected from SBI_00115 (WP_014172715.1), SBI_00631 (WP_014173231.1), SBI_01149 (WP_014173749.1) and SBI_01728 (WP_014174328.1); a Streptomyces avermitilis esterase selected from SAVERM_RS02860 (WP_010981907.1), SAVERM_RS04345 (WP_010036168.1), SAVERM_RS04550 (WP_107083239.1), and SAVERM RS23405(WP_010985956.1); a Streptomyces albus esterase selected from SLNWT_RS18180 (WP_078845043.1), SLNWT_RS12910 (WP_040249758.1) and SLNWT_RS12900 (WP_040249752.1); and a Bacillus subtilis esterase selected from BSU_08350 (NP_388716.1), BSU_24510 (NP_390331.1) and BSU_21740 (NP_390057.1).

37. The method of claim 27, wherein the enzyme is operably linked to downstream of an inducible promoter, and switching from a primary metabolism to a secondary metabolism is achieved by inducing expression.

38. A Streptomyces for producing a polyketide compound by fermentation, comprising at least one enzyme, wherein an expression level and/or activity of at least one enzyme in the Streptomyces that catalyzes an irreversible reaction of a β-oxidation pathway is enhanced compared with an original strain.

39. The Streptomyces of claim 38, wherein at least one enzyme that catalyzes an irreversible reaction of the β-oxidation pathway is provided downstream of an inducible promoter.

40. The Streptomyces of claim 39, wherein the enzyme that catalyzes an irreversible reaction of the β-oxidation pathway is selected from the group consisting of an acyl coenzyme A synthetase, an acyl-coenzyme A dehydrogenase, an acyl-coenzyme A hydratase, and any combination thereof.

41. The Streptomyces of claim 40, wherein the Streptomyces is a Streptomyces coelicolor, and the acyl coenzyme A synthetase is selected from the group consisting of SCO1330, SCO2131, SCO2444, SCO2561, SCO2720, SCO3436, SCO4006, SCO4503, SCO5983, SCO6196, SCO6552, SCO6790, SCO6968, SCO7244, SCO7329, SCO4383, and any combination thereof; the acyl-coenzyme A dehydrogenase is selected from the group consisting of SCO1690, SCO2774, SCO6787, and any combination thereof; and the acyl-coenzyme A hydratase is selected from SCO4384 and/or SCO6732.

42. The Streptomyces of claim 40, wherein the Streptomyces is a Streptomyces albus, the acyl coenzyme A synthetase is selected from the group consisting of SLNWT_0050, SLNWT_0304, SLNWT_0327, SLNWT_0598, SLNWT_0621, SLNWT_3453, SLNWT_4291, SLNWT_6199, SLNWT_6951, and any combination thereof; the acyl-coenzyme A dehydrogenase is SLNWT_4686; and the acyl-coenzyme A hydratase is selected from the group consisting of SLNWT_0723, SLNWT_0850, SLNWT_4292, SLNWT_6769, SLNWT_6771, and any combination thereof.

43. The Streptomyces of claim 40, wherein the Streptomyces is a Streptomyces venezuelae, the acyl coenzyme A synthetase is selected from the group consisting of SVEN_0294, SVEN_0876, SVEN_2231, SVEN_3097, SVEN_4199, SVEN_6078, SVEN_6188, SVEN_6773, SVEN_6774, SVEN_7224, and any combination thereof; the acyl-coenzyme A dehydrogenase is selected from SVEN_0520 and/or SVEN_1293; and the acyl-coenzyme A hydratase is selected from the group consisting of SVEN_0030, SVEN_0204, SVEN_0279, SVEN_1657, SVEN_4200, SVEN_5574, SVEN_5576, SVEN_6413, and any combination thereof.

44. The Streptomyces of claim 40, wherein the Streptomyces is a Streptomyces lividans, and the acyl coenzyme A synthetase is selected from the group consisting of SLIV_03075, SLIV_04410, SLIV_07155, SLIV_16515, SLIV_25480, SLIV_36365, and any combination thereof; the acyl-coenzyme A dehydrogenase is SLIV_29290; and the acyl-coenzyme A hydratase is selected from SLIV_16510 and/or SLIV_36115.

45. The Streptomyces of claim 40, wherein the Streptomyces is a Streptomyces avermitilis, and the acyl coenzyme A synthetase is selected from the group consisting of SAVERM_1258, SAVERM_1346, SAVERM_1603, SAVERM_2030, SAVERM_2279, SAVERM_377, SAVERM_3806, SAVERM_3864, SAVERM_5723, SAVERM_605, SAVERM_6612, and any combination thereof; the acyl-coenzyme A dehydrogenase is selected from the group consisting of SAVERM_1381, SAVERM_5280, SAVERM_6614, and any combination thereof; and the acyl-coenzyme A hydratase is selected from the group consisting of SAVERM_1245, SAVERM_1680, SAVERM_3863, SAVERM_6203, SAVERM_717, SAVERM_7216, and any combination thereof.

46. The Streptomyces of claim 40, wherein the Streptomyces is a Streptomyces rimosus, and the acyl coenzyme A synthetase is selected from the group consisting of an acyl coenzyme A synthetase having a NCBI registration number of WP_053803359.1, ELQ77730.1, WP_033034442.1, KOT44666.1, WP_033033106.1, and any combination thereof; the acyl-coenzyme A dehydrogenase is selected from the group consisting of an acyl coenzyme A dehydrogenase having a NCBI registration number of WP_125057199.1, WP_033031914.1, WP_030661846.1, WP_030634872.1, WP_030370993.1, and any combination thereof; and the acyl-coenzyme A hydratase is selected from an acyl-coenzyme A hydratase having a NCBI registration number of WP_030669923.1 and/or WP_125053679.1.

47. The Streptomyces of claim 40, wherein the Streptomyces is a Streptomyces bingchenggensis, and the acyl coenzyme A synthetase is selected from the group consisting of SBI_00524, SBI_02958, SBI_03178, SBI_04546, SBI_04871, SBI_06310, SBI_07635, SBI_08381, SBI_08662, SBI_09123, and any combination thereof; the acyl-coenzyme A dehydrogenase is selected from SBI_08383 and/or SBI_09842; and the acyl-coenzyme A hydratase is selected from the group consisting of SBI_01088, SBI_01673, SBI_01731, SBI_02642, SBI_04870, and any combination thereof.

48. The Streptomyces of claim 38, wherein the polyketide compound is selected from the group consisting of a type I polyketone compound, a type II polyketone compound, and a type III polyketone compound.

49. The Streptomyces bacterium of claim 48, wherein the polyketide is selected from the group consisting of actinomycin, jadomycin, avermectin, milbemycin, oxytetracycline and nemadectin.

50. The Streptomyces of claim 38, wherein a fatty acid moiety of a triacylglycerol is a fatty acid having a carbon number of 12-24.

51. The Streptomyces of claim 38, wherein the Streptomyces is selected from the group consisting of a Streptomyces coelicolor, a Streptomyces albus, a Streptomyces venezuelae, a Streptomyces lividans, a Streptomyces avermitilis, a Streptomyces rimosus, a Streptomyces hygroscopicus, a Streptomyces cyaneogriseus, and a Streptomyces bingchenggensis.

52. (canceled)


SEQUENCE LISTING (28 sequences)

SEQ ID NO: 1 - DNA, 49 bp, Artificial Sequence
  primer
aagcttgggctgcaggtcgactctagagttatcaccgcttgaacttggc

SEQ ID NO: 2 - DNA, 59 bp, Artificial Sequence
  primer
gacggctggggcgcgggtgcggtcactggggtcctcctgttgctcgactagtataatac

SEQ ID NO: 3 - DNA, 26 bp, Artificial Sequence
  primer
gtgaccgcacccgcgccccagccgtc

SEQ ID NO: 4 - DNA, 59 bp, Artificial Sequence
  primer
acatgattacgaattcgatatcgcgcgcggccgcggatctcaggggcgcgctccgtacc

SEQ ID NO: 5 - DNA, 52 bp, Artificial Sequence
  primer
gccaagttcaagcggtgataatctagacgctccctgcccgctatggtgacga

SEQ ID NO: 6 - DNA, 49 bp, Artificial Sequence
  primer
ccctgatgataagcattacgatatcgaattcgtaatcatgtcatagctg

SEQ ID NO: 7 - DNA, 47 bp, Artificial Sequence
  primer
cagtgccaagcttgggctgcaggagaacggtccggcgattgtcctcg

SEQ ID NO: 8 - DNA, 50 bp, Artificial Sequence
  primer
cgagtgggtcctcgtccagtacgggatccaggagttcctgtacgcccacc

SEQ ID NO: 9 - DNA, 33 bp, Artificial Sequence
  primer
tcctggatcccgtactggacgaggacccactcg

SEQ ID NO: 10 - DNA, 49 bp, Artificial Sequence
  primer
ctatgacatgattacgaattcgatatcgatgaaactgcgcgcggtcgtc

SEQ ID NO: 11 - DNA, 31 bp, Artificial Sequence
  primer
catcgatatcgaattcgtaatcatgtcatag

SEQ ID NO: 12 - DNA, 30 bp, Artificial Sequence
  primer
gttctcctgcagcccaagcttggcactggc

SEQ ID NO: 13 - DNA, 22 bp, Artificial Sequence
  primer
agtaggcgcgcagttcctccag

SEQ ID NO: 14 - DNA, 27 bp, Artificial Sequence
  primer
tcccgtccggacggcgctggacctacg

SEQ ID NO: 15 - DNA, 50 bp, Artificial Sequence
  primer
atctagcggaacggatctagagatgtgaccgcacccgcgccccagccgtc

SEQ ID NO: 16 - DNA, 48 bp, Artificial Sequence
  primer
ttccatcgccgcttcatgatgaattctcgccgcggccgataccggtgc

SEQ ID NO: 17 - DNA, 19 bp, Artificial Sequence
  primer
ggcatctgggcggtcaact

SEQ ID NO: 18 - DNA, 19 bp, Artificial Sequence
  primer
gctgctcttgtgggcgagg

SEQ ID NO: 19 - DNA, 23 bp, Artificial Sequence
  primer
tgtccgccctccgctccgtgtcc

SEQ ID NO: 20 - DNA, 21 bp, Artificial Sequence
  primer
tccaggaccgtgtcgccgtag

SEQ ID NO: 21 - DNA, 20 bp, Artificial Sequence
  primer
gtgcagctcctgtacacctc

SEQ ID NO: 22 - DNA, 20 bp, Artificial Sequence
  primer
ctcaggtactcgtgcaccag

SEQ ID NO: 23 - DNA, 20 bp, Artificial Sequence
  primer
cagaccgtctccctcaactc

SEQ ID NO: 24 - DNA, 20 bp, Artificial Sequence
  primer
cctccttcaccatcagctcg

SEQ ID NO: 25 - DNA, 20 bp, Artificial Sequence
  primer
catccagaaccaccgcatca

SEQ ID NO: 26 - DNA, 20 bp, Artificial Sequence
  primer
tcagcgaggagaggtcgtag

SEQ ID NO: 27 - DNA, 20 bp, Artificial Sequence
  primer
agagcggcggttacaagatc

SEQ ID NO: 28 - DNA, 18 bp, Artificial Sequence
  primer
cacgatccgttccccgag